(19) United States
(12) Patent Application Publication
(10) Pub. No.: US 2019/0054000 A1
(43) Pub. Date: Feb. 21, 2019

(54) Actinic Light Curable Dip System

(71) Applicant: MYCONE DENTAL SUPPLY CO., INC., Gibbstown, NJ (US)

(72) Inventors: Kendra GOUSE, Philadelphia, PA (US); Kevin SHERAN, Haddonfield, NJ (US); Megan SHARP, Gloucester City, NJ (US)

(73) Assignee: MYCONE DENTAL SUPPLY CO., INC., Gibbstown, NJ (US)

(21) Appl. No.: 16/108,091

(22) Filed: Aug. 21, 2018

Related U.S. Application Data
(60) Provisional application No. 62/547,953, filed on Aug. 21, 2017.

(51) Int. Cl. A61K 8/81; A61K 8/04; A61K 8/02; A61K 8/58; A61Q 3/02; C09D 4/06

(52) U.S. Cl. CPC A61K 8/8152; A61K 8/042; A61K 8/022; A61K 8/025; A61K 8/585; A61Q 3/02; C08J 3/24; A61K 2800/81; A61K 2800/95; A61K 2800/884; A61K 2800/412; A61K 2800/43; C09D 4/06

(57) ABSTRACT

An actinic light curable dip gel comprises an acrylic resin selected from the group consisting of a poly(C₁₋₁₂alkyl(meth)acrylate) polymer, an acrylate functional acrylic resin, and mixtures thereof; a monomer; at least one photoinitiator; and a solvent. A layer of this actinic light curable dip gel is applied onto a nail, followed by an application of a polymer powder onto the nail; and exposing the nail to actinic light. The steps of applying the gel layer and the polymer powder may be repeated several times prior to exposing the nail to actinic light. The use of the actinic light curable dip gel has several advantages over the traditional dip system.

### CROSS-REFERENCE TO RELATED APPLICATIONS

This application is related to and claims priority to U.S. Provisional Patent Application No. 62/547,953, filed on 21 Aug. 2017, and entitled “Actinic Light Curable Dip System” which is incorporated by reference herein in its entirety.

### FIELD OF THE INVENTION

The present invention is directed to a formulation and a method of forming a nail coating. More particularly, the invention relates to the field of actinic light curable dip systems suitable for use with polymeric powders.

### DESCRIPTION OF RELATED TECHNOLOGY

In a traditional two-part system, a monomer liquid is combined with a polymer powder to obtain a dough. The dough is then transferred onto a client's nail, and must be quickly shaped into a desired shape on a nail before the dough hardens. This procedure requires a high level of skill. The final nail covering is thick and thus is typically filed down after shaping. This filing step takes time and produces a large amount of dust.

In a traditional dip system, a nail is coated with an adhesive, such as a cyanoacrylate, which is then dipped into a powder or sprinkled with the powder before the adhesive cures. The powder adheres to the uncured adhesive. This method of applying of the adhesive followed by dipping or sprinkling is typically repeated several times to obtain the desired aesthetic effect. An activator is then applied to the multilayered coating so that the cyanoacrylate adhesive completely cures. An optional filing of the nail or multilayered coating may now take place. Finally, a top coat is applied to protect and make the nail coating aesthetically pleasing.

A problem with the existing the traditional dip system is the number of liquid components required. For example, some commercially available dip systems have an adhesive coat (such as a cyanoacrylate), an activator coat (or initiator, typically N,N-dimethyl-p-toluidine or DMPT), and an air dry top coat. Multiple top sealer coats are required to achieve the final nail covering.

Another common problem is the relatively short open time. If a nail technician waits more than one minute or so after applying the adhesive to dip the nail in the powder or sprinkle it with powder, the powder won't adhere to the adhesive.

Yet another common problem with the traditional dip system is the short stability/shelf life of the ethyl cyanoacrylate adhesive. This problem may be traced to cross-contamination involving the brush used to apply the cyanoacrylate adhesive: the brush picks up some powder applied in a previous cycle and carries it back to the bottle of adhesive, making the brush unusable due to polymerization of the adhesive that remains on it. A brush cleaner liquid is typically included among the various liquids supplied with traditional dip kits, but it does not eliminate the problem. The act of using the cyanoacrylate adhesive tends to glue the brush and cap to the container rendering the contents unavailable and therefore wasted. The stability/shelf life of the cyanoacrylate adhesive tends to be short potentially rendering even an unopened container useless.

Although many advances in the art of formulating nail polish compositions have been made, many more challenges remain.

### SUMMARY OF THE INVENTION

The present invention relates to an actinic light curable dip gel. This actinic light curable dip gel is designed to be used with a powder to generate a nail coating. The nail coating may also comprise a base coat or a primer, and a top coat.

The actinic light curable dip gel comprises a film-forming a resin; a monomer; a photoinitiator; and optionally, a solvent, and/or a crosslinker.

More specifically, the present invention is directed to an actinic light curable dip gel comprising: a film forming resin selected from the group consisting of poly(C₁₋₁₂alkyl(meth)acrylate) polymer, an acrylate functional acrylic resin, poly(meth)acrylate oligomer, styrene polymer, styrene divinylbenzene polymer, urethane polymer, cellulose acetate butyrate, cellulose acetate propionate, nitrocellulose, and mixtures thereof; a monomer; and a photoinitiator. Examples of an acrylic resin include a poly(alkyl(meth)acrylate) polymer, wherein the alkyl groups comprise one to twelve carbon atoms, an acrylate functional acrylic resin, and mixtures thereof.

An acrylate functional acrylic resin is an acrylic resin obtained by reacting an acrylic acid with a poly(C₁₋₁₂alkyl (meth)acrylate) polymer, poly(C₁₋₁₂alkyl (meth)acrylate) copolymer, or poly(C₁₋₁₂alkyl (meth)acrylate)/styrene copolymer.

The actinic light curable dip gel may comprise between 0 wt % and about 50 wt % of the poly(C₁₋₁₂alkyl(meth)acrylate) polymer. The actinic light curable dip gel may comprise between 0 wt % and about 50 wt % of the acrylate functional acrylic resin. The actinic light curable dip gel may comprises between 0 wt % and about 50 wt % of the poly(meth)acrylate oligomer.

The actinic light curable dip gel of the present invention comprises one or more monomers. An example of such monomers includes (meth)acrylate monomers.

The actinic light curable dip gel of the present invention is designed to be used with a powder to generate a nail coating. The mixture of the dip gel and the powder also comprises a photoinitiator. The photoinitiator is selected so that it is activated by photons of the wavelength associated with actinic light of, for example, a UV light lamp.

The actinic light curable dip gel of the present invention may also comprise a solvent. Under one embodiment, the solvent is selected from the group consisting of C₁₋₄ alkyl acetate, C₁₋₃ alcohol, C₃₋₅ ketone, and a mixture thereof.

Under one embodiment the actinic light curable dip gel further comprises a crosslinker. Under another embodiment the monomer liquid comprises two or more different compounds that are crosslinkers. A cross-linker is a multifunctional monomer. Examples of crosslinkers include diacrylates, triacrylates, tetraacrylates, pentaacrylates and higher acrylates.

Under one embodiment the actinic light curable dip gel further comprises a silane coupling agent. The silane coupling agent is an organofunctional silane which comprises a reactive functional group.

Under one embodiment, the actinic light curable dip gel comprises an acrylic resin and a monomer, but very little or no oligomer.

The actinic light curable dip gel optionally comprises additional ingredients, such a colorant or a pigment.

The present invention is also directed to a general method of forming a nail cover comprising the steps of applying a layer of the actinic light curable dip gel onto a nail, applying a powder onto the nail, and exposing the nail to actinic light, wherein the steps may be carried out in any order. This method may be exemplified by any of the at least four methods of forming a nail cover.

Under one method of forming a nail cover, the method of forming a nail cover comprises the steps of (a)(i) applying a layer of the actinic light curable dip gel onto a nail; (ii) introducing a powder onto the nail; (iii) repeating steps (i) and (ii) 0 to 5 times; and (b) exposing the nail to actinic light.

In this method, the steps (a)(i) and (a)(ii) are repeated zero to five times, and then the nail is exposed to the actinic light.

Under one embodiment, steps (a)(i) and (a)(ii) are done only once. The method of forming a nail cover under this embodiment comprises the steps of applying a layer of the actinic light curable dip gel onto a nail; dipping the nail into a polymer powder; and exposing the nail to actinic light.

In step (a)(i) of the method of forming a nail cover, the actinic light curable dip gel is applied onto the nail.

After the transfer of the actinic light curable dip gel onto the nail, the gel is spread out to make a layer of the actinic light curable dip gel. The nail is covered by a layer of the actinic light curable dip gel, and upon application of the polymer powder onto the nail, the polymer powder sticks to the gel. After the polymer powder sticks to the gel, the gel interacts with the polymer powder to create a uniform layer.

The application of the polymer powder onto the nail occurs prior to curing of the actinic light curable dip gel. The period of time from the application of the actinic light curable dip gel to the beginning of the application of the polymer powder may be as little as 5 seconds, or as long as 10 minutes.

Under one embodiment, the powder is similar to, or is the same as, the polymer powder that is typically used in the acrylic nail industry. When mixed with the actinic light curable dip gel, the actinic light curable dip gel is a continuous phase, and the polymer powder is a discontinuous phase.

Under one embodiment, the powder comprises a polyalkyl(meth)acrylate, or a mixture thereof, wherein the alkyl groups comprise one to twelve carbon atoms. Under another embodiment, the powder does not chemically react, or does not substantially chemically react, with the actinic light curable dip gel. The nail cover, once cured, is a solid comprising at least two distinct phases: a continuous phase, and a discontinuous phase.

The present invention is also directed to a method of forming a nail cover comprising the steps of (a)(i) applying a layer of the actinic light curable dip gel onto a nail; (ii) applying onto the nail a powder selected from the group consisting of: special effects pigments, spherical aliphatic polyurethane beads, micronized polyethylene and PTFE, micronized polypropylene wax, silicone resin powder, styrene polymer, styrene copolymer, glass beads, nylon beads and polyethylene terephthalate based effect pigments, and mixtures thereof; (iii) repeating steps (i) and (ii) 0 to 5 times; and (b) exposing the nail to actinic light.

The present invention is also directed to a method of forming a nail cover comprising the steps of (a) applying a layer of the actinic light curable dip gel onto a nail; (b) applying onto the nail a powder selected from the group consisting of: special effects pigment, spherical aliphatic polyurethane beads, micronized polyethylene and PTFE, micronized polypropylene wax, silicone resin powder, styrene polymer, styrene copolymer, glass beads, nylon beads and polyethylene terephthalate based effect pigments, and mixtures thereof; (c) exposing the nail to actinic light; and (d) repeating steps (a) to (c) 0 to 5 times.

After a desired number of repetitions, the nail is exposed to actinic light to cure the dip gel. A suitable actinic light may be natural sunlight. Another suitable actinic light may be a UV light lamp, such as a 36-watt lamp commonly used in many nail salons. Such a UV light lamp may operate at any wavelength required to cure the photopolymerizable composition, such as between 320 nm and 420 nm range at sufficient enough strength to cure the composition of the present invention.

The present invention is also directed to a method of forming a nail cover comprising the steps of (a) applying a layer of the actinic light curable dip gel onto a nail; (b) applying a powder onto the nail; (c) exposing the nail to actinic light; and (d) repeating steps (a) to (c) 0 to 5 times. This method is particularly suitable for novice nail technicians.

The present invention is also directed to a method of forming a nail cover comprising the steps of (a) applying a layer of the actinic light curable dip gel onto a nail; (b) exposing the nail to actinic light; (c) applying a powder onto the nail; and (d) repeating steps (a) to (c) 0 to 5 times.

The present invention is also directed to a method of forming a nail cover comprising the steps of (a)(i) applying a layer of the actinic light curable dip gel onto a nail; (ii) applying a powder onto the nail; (iii) repeating steps (i) and (ii) 0 to 5 times; (b) applying a top coat; and (c) exposing the nail to actinic light.

For each of the four methods disclosed above, additional steps may be performed by the technician to obtain the desired nail covering. These steps may follow the steps discussed above, or they may precede the steps discussed above, or they may occur between any of the steps discussed above.

Under one embodiment, prior to the application of the first layer of the actinic light curable dip gel onto a nail, the nail is coated with a nail polish primer or a nail polish base coat.

Under one embodiment, after the exposure of the nail to the actinic light, the nail coating is treated further. One such step is filing and/or buffing the nail until the surface is smooth. Another such step is the application of a top coat.

The invention is defined by at least twenty-seven aspects.

In the first aspect, the invention relates to an actinic light curable dip gel comprising: a film forming resin selected from the group consisting of poly(C₁₋₁₂alkyl(meth)acrylate) polymer, an acrylate functional acrylic resin, poly(meth)acrylate oligomer, styrene polymer, styrene divinylbenzene polymer, urethane polymer, cellulose acetate butyrate, cellulose acetate propionate, nitrocellulose, and mixtures thereof; a (meth)acrylate monomer; and one or more photoinitiators.

In the second aspect, the invention relates to an actinic light curable dip gel comprising: a film forming resin selected from the group consisting of poly(C₁₋₁₂alkyl(meth)acrylate) polymer, an acrylate functional acrylic resin, poly(meth)acrylate oligomer, styrene polymer, styrene divinylbenzene polymer, urethane polymer, cellulose acetate butyrate, cellulose acetate propionate, nitrocellulose, and mixtures thereof; a (meth)acrylate monomer; and one or more photoinitiators, wherein the film forming resin is selected from the group consisting of a poly(C₁₋₁₂alkyl(meth)acrylate) polymer, an acrylate functional acrylic resin, and mixtures thereof.

In the third aspect, the invention relates to an actinic light curable dip gel comprising: a film forming resin selected from the group consisting of poly(C₁₋₁₂alkyl(meth)acrylate) polymer, an acrylate functional acrylic resin, poly(meth)acrylate oligomer, styrene polymer, styrene divinylbenzene polymer, urethane polymer, cellulose acetate butyrate, cellulose acetate propionate, nitrocellulose, and mixtures thereof; a (meth)acrylate monomer; and one or more photoinitiators; wherein the actinic light curable dip gel comprises between 0 wt % and about 50 wt % of the poly(C₁₋₁₂alkyl(meth)acrylate) polymer.

In the fourth aspect, the invention relates to an actinic light curable dip gel comprising: a film forming resin selected from the group consisting of poly(C₁₋₁₂alkyl(meth)acrylate) polymer, an acrylate functional acrylic resin, poly(meth)acrylate oligomer, styrene polymer, styrene divinylbenzene polymer, urethane polymer, cellulose acetate butyrate, cellulose acetate propionate, nitrocellulose, and mixtures thereof; a (meth)acrylate monomer; and one or more photoinitiators; wherein the actinic light curable dip gel comprises between 0 wt % and about 50 wt % of the acrylate functional acrylic resin.

In the fifth aspect, the invention relates to an actinic light curable dip gel comprising: a film forming resin selected from the group consisting of poly(C₁₋₁₂alkyl(meth)acrylate) polymer, an acrylate functional acrylic resin, poly(meth)acrylate oligomer, styrene polymer, styrene divinylbenzene polymer, urethane polymer, cellulose acetate butyrate, cellulose acetate propionate, nitrocellulose, and mixtures thereof; a (meth)acrylate monomer; and one or more photoinitiators; wherein the actinic light curable dip gel comprises between 0 wt % and about 50 wt % of the poly(meth)acrylate oligomer.

In the sixth aspect, the invention relates to an actinic light curable dip gel comprising: a film forming resin selected from the group consisting of poly(C₁₋₁₂alkyl(meth)acrylate) polymer, an acrylate functional acrylic resin, poly(meth)acrylate oligomer, styrene polymer, styrene divinylbenzene polymer, urethane polymer, cellulose acetate butyrate, cellulose acetate propionate, nitrocellulose, and mixtures thereof; a (meth)acrylate monomer; and one or more photoinitiators; wherein at least one of the one or more photoinitiators is selected from the group consisting of benzyl ketone, monomeric hydroxyl ketone, polymeric hydroxyl ketone, α-amino ketone, acyl phosphine oxide, metallocene, benzophenone, camphor quinone, and benzophenone derivatives.

In the seventh aspect, the invention relates to an actinic light curable dip gel comprising: a film forming resin selected from the group consisting of poly(C₁₋₁₂alkyl(meth)acrylate) polymer, an acrylate functional acrylic resin, poly(meth)acrylate oligomer, styrene polymer, styrene divinylbenzene polymer, urethane polymer, cellulose acetate butyrate, cellulose acetate propionate, nitrocellulose, and mixtures thereof; a (meth)acrylate monomer; and one or more photoinitiators; wherein at least one of the one or more photoinitiators is selected from the group consisting of 1-hydroxy-cyclohexylphenylketone; benzophenone; camphor quinone; 2-benzyl-2-(dimethylamino)-1-(4-(4-morphorlinyl)phenyl)-1-butanone; 2,2-dimethoxy-2-phenyl acetophenone; 2-methyl-1-(4-methylthio)phenyl-2-(4-morphorlinyl)-1-propanone; 2,4,6-trimethylbenzoyldiphenyl-phosphine oxide; bis(2,4,6-trimethyl benzoyl)phenyl wephosphine oxide; diphenyl-(2,4,6-trimethylbenzoyl) phosphine oxide; bis(2,6-dimethoxybenzoyl-2,4,4-trimethyl pentyl)phosphine oxide; 2-hydroxy-2-methyl-1-phenyl-propan-1-one; phenyl bis(2,4,6-trimethylbenzoyl) phosphine oxide; benzyl-dimethylketal; isopropylthioxanthone; bis(η⁵-2,4-cyclopentadien-1-yl)bis[2,6-difluoro-3-(1H-pyrrol-1-yl)phenyl]titanium), and mixtures of any of the foregoing.

In the eighth aspect, the invention relates to an actinic light curable dip gel comprising: a film forming resin selected from the group consisting of poly(C₁₋₁₂alkyl(meth)acrylate) polymer, an acrylate functional acrylic resin, poly(meth)acrylate oligomer, styrene polymer, styrene divinylbenzene polymer, urethane polymer, cellulose acetate butyrate, cellulose acetate propionate, nitrocellulose, and mixtures thereof; a (meth)acrylate monomer; and one or more photoinitiators; further comprising a solvent selected from the group consisting of C₁₋₄ alkyl acetate, C₁₋₃ alcohol, C₃₋₅ ketone, and mixtures thereof.

In the ninth aspect, the invention relates to an actinic light curable dip gel comprising: a film forming resin selected from the group consisting of poly(C₁₋₁₂alkyl(meth)acrylate) polymer, an acrylate functional acrylic resin, poly(meth)acrylate oligomer, styrene polymer, styrene divinylbenzene polymer, urethane polymer, cellulose acetate butyrate, cellulose acetate propionate, nitrocellulose, and mixtures thereof; a (meth)acrylate monomer; and one or more photoinitiators; further comprising at least one crosslinker.

In the tenth aspect, the invention relates to an actinic light curable dip gel comprising: a film forming resin selected from the group consisting of poly(C₁₋₁₂alkyl(meth)acrylate) polymer, an acrylate functional acrylic resin, poly(meth)acrylate oligomer, styrene polymer, styrene divinylbenzene polymer, urethane polymer, cellulose acetate butyrate, cellulose acetate propionate, nitrocellulose, and mixtures thereof; a (meth)acrylate monomer; and one or more photoinitiators, further comprising a crosslinker selected from the group consisting of tri(meth)acrylate, tetra(meth)acrylate, penta(meth)acrylate, ethylene glycol dimethylacrylate, EGDMA, di(ethylene glycol) dimethylacrylate, di-EGDMA, tri-(ethylene glycol) dimethylacrylate, and a mixture thereof.

In the eleventh aspect, the invention relates to an actinic light curable dip gel comprising: a film forming resin selected from the group consisting of poly(C₁₋₁₂alkyl(meth)acrylate) polymer, an acrylate functional acrylic resin, poly(meth)acrylate oligomer, styrene polymer, styrene divinylbenzene polymer, urethane polymer, cellulose acetate butyrate, cellulose acetate propionate, nitrocellulose, and mixtures thereof; a (meth)acrylate monomer; and one or more photoinitiators; further comprising a crosslinker selected from the group consisting of trimethylol propane tri(meth)acrylate, ethoxylated glycerin tri(meth)acrylate, ethoxylated trimethylolpropane tri(meth)acrylate, ditrimethylol propane tetra(meth)acrylate, pentaerythritol tri(meth)acrylate, pentaerythritol tetra(meth)acrylate, propoxylated pentaerythritol tetra(meth)acrylate, ethoxylated pentaerythritol tetra(meth)acrylate, dipentaerythritol hexa(meth)acrylate, dipentaerythritol penta(meth)acrylate and ethoxylated iscyanuric acid tri(meth)acrylates.

In the twelfth aspect, the invention relates to an actinic light curable dip gel comprising: a film forming resin selected from the group consisting of poly(C₁₋₁₂alkyl(meth)acrylate) polymer, an acrylate functional acrylic resin, poly(meth)acrylate oligomer, styrene polymer, styrene divinylbenzene polymer, urethane polymer, cellulose acetate butyrate, cellulose acetate propionate, nitrocellulose, and mixtures thereof; a (meth)acrylate monomer; and one or more photoinitiators; further comprising a silane coupling agent.

In the thirteenth aspect, the invention relates to an actinic light curable dip gel comprising: a film forming resin selected from the group consisting of poly(C₁₋₁₂alkyl(meth)acrylate) polymer, an acrylate functional acrylic resin, poly(meth)acrylate oligomer, styrene polymer, styrene divinylbenzene polymer, urethane polymer, cellulose acetate butyrate, cellulose acetate propionate, nitrocellulose, and mixtures thereof; a (meth)acrylate monomer; and one or more photoinitiators; wherein the gel is stable in a dark container at 49° C. for four months, or at 65° C. for 2 weeks.

In the fourteenth aspect, the invention relates to a method of forming a nail cover comprising the steps of applying a layer of an actinic light curable dip gel comprising: a film forming resin selected from the group consisting of poly(C₁₋₁₂alkyl(meth)acrylate) polymer, an acrylate functional acrylic resin, poly(meth)acrylate oligomer, styrene polymer, styrene divinylbenzene polymer, urethane polymer, cellulose acetate butyrate, cellulose acetate propionate, nitrocellulose, and mixtures thereof a (meth)acrylate monomer; and one or more photoinitiators; onto a nail, applying a powder onto the nail, and exposing the nail to actinic light, wherein the steps may be carried out in any order.

In the fifteenth aspect, the invention relates to a method of forming a nail cover comprising the steps of applying a layer of an actinic light curable dip gel comprising: a film forming resin selected from the group consisting of poly(C₁₋₁₂alkyl(meth)acrylate) polymer, an acrylate functional acrylic resin, poly(meth)acrylate oligomer, styrene polymer, styrene divinylbenzene polymer, urethane polymer, cellulose acetate butyrate, cellulose acetate propionate, nitrocellulose, and mixtures thereof a (meth)acrylate monomer; and one or more photoinitiators; onto a nail, applying a powder onto the nail, and exposing the nail to actinic light, wherein the steps may be carried out in any order, wherein the powder is selected from an organic powder, inorganic powder, and a mixture thereof.

In the sixteenth aspect, the invention relates to a method of forming a nail cover comprising the steps of applying a layer of an actinic light curable dip gel comprising: a film forming resin selected from the group consisting of poly(C₁₋₁₂alkyl(meth)acrylate) polymer, an acrylate functional acrylic resin, poly(meth)acrylate oligomer, styrene polymer, styrene divinylbenzene polymer, urethane polymer, cellulose acetate butyrate, cellulose acetate propionate, nitrocellulose, and mixtures thereof; a (meth)acrylate monomer; and one or more photoinitiators; onto a nail, applying a powder onto the nail, and exposing the nail to actinic light, wherein the steps may be carried out in any order; wherein the powder comprises a polymer powder selected from the group consisting of acrylic polymer, styrenic polymer, urethane polymer, and mixtures thereof.

In the seventeenth aspect, the invention relates to a method of forming a nail cover comprising the steps of applying a layer of an actinic light curable dip gel comprising: a film forming resin selected from the group consisting of poly(C₁₋₁₂alkyl(meth)acrylate) polymer, an acrylate functional acrylic resin, poly(meth)acrylate oligomer, styrene polymer, styrene divinylbenzene polymer, urethane polymer, cellulose acetate butyrate, cellulose acetate propionate, nitrocellulose, and mixtures thereof; a (meth)acrylate monomer; and one or more photoinitiators; onto a nail, applying a powder onto the nail, and exposing the nail to actinic light, wherein the steps may be carried out in any order; wherein the powder comprises a polymer powder selected from the group consisting of acrylic polymer, styrenic polymer, urethane polymer, and mixtures thereof; wherein the polymer powder is crosslinked.

In the eighteenth aspect, the invention relates to a method of forming a nail cover comprising the steps of applying a layer of an actinic light curable dip gel comprising: a film forming resin selected from the group consisting of poly(C₁₋₁₂alkyl(meth)acrylate) polymer, an acrylate functional acrylic resin, poly(meth)acrylate oligomer, styrene polymer, styrene divinylbenzene polymer, urethane polymer, cellulose acetate butyrate, cellulose acetate propionate, nitrocellulose, and mixtures thereof; a (meth)acrylate monomer; and one or more photoinitiators; onto a nail, applying a powder onto the nail, and exposing the nail to actinic light, wherein the steps may be carried out in any order; wherein the powder comprises particles selected from the group consisting of: a special effects pigment, spherical aliphatic polyurethane beads, micronized polyethylene and PTFE, micronized polypropylene wax, silicone resin powder, styrene polymer, styrene polymer, styrene copolymer, glass beads, nylon beads and polyethylene terephthalate based effect pigments, and mixtures thereof.

In the nineteenth aspect, the invention relates to a method of forming a nail cover comprising the steps of applying a layer of an actinic light curable dip gel comprising: a film forming resin selected from the group consisting of poly(C₁₋₁₂alkyl(meth)acrylate) polymer, an acrylate functional acrylic resin, poly(meth)acrylate oligomer, styrene polymer, styrene divinylbenzene polymer, urethane polymer, cellulose acetate butyrate, cellulose acetate propionate, nitrocellulose, and mixtures thereof; a (meth)acrylate monomer; and one or more photoinitiators; onto a nail, applying a powder onto the nail, and exposing the nail to actinic light, wherein the steps may be carried out in any order; wherein the powder comprises particles selected from the group consisting of: a special effects pigment, spherical aliphatic polyurethane beads, micronized polyethylene and PTFE, micronized polypropylene wax, silicone resin powder, styrene polymer, styrene polymer, styrene copolymer, glass beads, nylon beads and polyethylene terephthalate based effect pigments, and mixtures thereof; wherein the powder comprises special effects pigment selected from the group consisting of mica, aluminum, calcium borosilicate, titanium, synthetic mica, and a mixture thereof.

In the twentieth aspect, the invention relates to a method of forming a nail cover comprising the steps of applying a layer of an actinic light curable dip gel comprising: a film forming resin selected from the group consisting of poly(C₁₋₁₂alkyl(meth)acrylate) polymer, an acrylate functional acrylic resin, poly(meth)acrylate oligomer, styrene polymer, styrene divinylbenzene polymer, urethane polymer, cellulose acetate butyrate, cellulose acetate propionate, nitrocellulose, and mixtures thereof; a (meth)acrylate monomer; and one or more photoinitiators; onto a nail, applying a powder onto the nail, and exposing the nail to actinic light, wherein the steps may be carried out in any order; wherein the powder comprises particles selected from the group consisting of: a special effects pigment, spherical aliphatic polyurethane beads, micronized polyethylene and PTFE, micronized polypropylene wax, silicone resin powder, styrene polymer, styrene polymer, styrene copolymer, glass beads, nylon beads and polyethylene terephthalate based effect pigments, and mixtures thereof; wherein the special effects pigment is selected from the group consisting a metal, a metal oxide, a main group oxide, a metal/main group oxide, and a mixture thereof.

In the twenty-first aspect, the invention relates to a method of forming a nail cover comprising the steps of applying a layer of an actinic light curable dip gel comprising: a film forming resin selected from the group consisting of poly(C₁₋₁₂alkyl(meth)acrylate) polymer, an acrylate functional acrylic resin, poly(meth)acrylate oligomer, styrene polymer, styrene divinylbenzene polymer, urethane polymer, cellulose acetate butyrate, cellulose acetate propionate, nitrocellulose, and mixtures thereof; a (meth)acrylate monomer; and one or more photoinitiators; onto a nail, applying a powder onto the nail, and exposing the nail to actinic light, wherein the steps may be carried out in any order; wherein the powder is spherical.

In the twenty-second aspect, the invention relates to a method of forming a nail cover comprising the steps of applying a layer of an actinic light curable dip gel comprising: a film forming resin selected from the group consisting of poly(C₁₋₁₂alkyl(meth)acrylate) polymer, an acrylate functional acrylic resin, poly(meth)acrylate oligomer, styrene polymer, styrene divinylbenzene polymer, urethane polymer, cellulose acetate butyrate, cellulose acetate propionate, nitrocellulose, and mixtures thereof; a (meth)acrylate monomer; and one or more photoinitiators; onto a nail, applying a powder onto the nail, and exposing the nail to actinic light, wherein the steps may be carried out in any order; wherein the mean particle size of poly(methyl methacrylate) particles is less than about 100 micrometers.

In the twenty-third aspect, the invention relates to a method of forming a nail cover comprising the steps of applying a layer of an actinic light curable dip gel comprising: a film forming resin selected from the group consisting of poly(C₁₋₁₂alkyl(meth)acrylate) polymer, an acrylate functional acrylic resin, poly(meth)acrylate oligomer, styrene polymer, styrene divinylbenzene polymer, urethane polymer, cellulose acetate butyrate, cellulose acetate propionate, nitrocellulose, and mixtures thereof; a (meth)acrylate monomer; and one or more photoinitiators; onto a nail, applying a powder onto the nail, and exposing the nail to actinic light, wherein the steps may be carried out in any order; wherein the first step is preceded by a step of applying a base coat layer, or a primer layer.

In the twenty-fourth aspect, the invention relates to a method of forming a nail cover comprising the steps of (a)(i) applying a layer of the actinic light curable dip gel of comprising: a film forming resin selected from the group consisting of poly(C₁₋₁₂alkyl(meth)acrylate) polymer, an acrylate functional acrylic resin, poly(meth)acrylate oligomer, styrene polymer, styrene divinylbenzene polymer, urethane polymer, cellulose acetate butyrate, cellulose acetate propionate, nitrocellulose, and mixtures thereof; a (meth)acrylate monomer; and one or more photoinitiators, onto a nail; (ii) applying a powder onto the nail; (iii) repeating steps (i) and (ii) 0 to 5 times; and (b) exposing the nail to actinic light.

In the twenty-fifth aspect, the invention relates to a method of forming a nail cover comprising the steps of (a) applying a layer of an actinic light curable dip gel comprising: a film forming resin selected from the group consisting of poly(C₁₋₁₂alkyl(meth)acrylate) polymer, an acrylate functional acrylic resin, poly(meth)acrylate oligomer, styrene polymer, styrene divinylbenzene polymer, urethane polymer, cellulose acetate butyrate, cellulose acetate propionate, nitrocellulose, and mixtures thereof; a (meth)acrylate monomer; and one or more photoinitiators, onto a nail; (b) applying a powder onto the nail; (c) exposing the nail to actinic light; and (d) repeating steps (a) to (c) 0 to 5 times.

In the twenty-sixth aspect, the invention relates to a method of forming a nail cover comprising the steps of (a) applying a layer of an actinic light curable dip gel comprising: a film forming resin selected from the group consisting of poly(C₁₋₁₂alkyl(meth)acrylate) polymer, an acrylate functional acrylic resin, poly(meth)acrylate oligomer, styrene polymer, styrene divinylbenzene polymer, urethane polymer, cellulose acetate butyrate, cellulose acetate propionate, nitrocellulose, and mixtures thereof; a (meth)acrylate monomer; and one or more photoinitiators, onto a nail; (b) exposing the nail to actinic light; (c) applying a powder onto the nail; and (d) repeating steps (a) to (c) 0 to 5 times.

In the twenty-seventh aspect, the invention relates to a method of forming a nail cover comprising the steps of (a)(i) applying a layer of an actinic light curable dip gel comprising: a film forming resin selected from the group consisting of poly(C₁₋₁₂alkyl(meth)acrylate) polymer, an acrylate functional acrylic resin, poly(meth)acrylate oligomer, styrene polymer, styrene divinylbenzene polymer, urethane polymer, cellulose acetate butyrate, cellulose acetate propionate, nitrocellulose, and mixtures thereof; a (meth)acrylate monomer; and one or more photoinitiators, onto a nail; (ii) applying a powder onto the nail; (iii) repeating steps (i) and (ii) 0 to 5 times; (b) applying a top coat; and (c) exposing the nail to actinic light.

### DETAILED DESCRIPTION OF THE INVENTION

For illustrative purposes, the principles of the present invention are described by referencing various exemplary embodiments thereof. Although certain embodiments of the invention are specifically described herein, one of ordinary skill in the art will readily recognize that the same principles are equally applicable to, and can be employed in other apparatuses and methods. Before explaining the disclosed embodiments of the present invention in detail, it is to be understood that the invention is not limited in its application to the details of any particular embodiment shown. The terminology used herein is for the purpose of description and not of limitation. Further, although certain methods are described with reference to certain steps that are presented herein in certain order, in many instances, these steps may be performed in any order as may be appreciated by one skilled in the art, and the methods are not limited to the particular arrangement of steps disclosed herein.

As used herein and in the appended claims, the singular forms “a”, “an”, and “the” include plural references unless the context clearly dictates otherwise. The singular form of any class of the ingredients refers not only to one chemical species within that class, but also to a mixture of those chemical species; for example, the term “solvent” in the singular form, may refer to a mixture of compounds each of which is also a solvent. The terms “a” (or “an”), “one or more” and “at least one” may be used interchangeably herein. The terms “comprising”, “including”, and “having” may be used interchangeably.

The abbreviations and symbols as used herein, unless indicated otherwise, take their ordinary meaning. The term “wt %” means percent by weight. The abbreviation “UV” stands for ultraviolet. Symbols “μm” and “nm” refer to micrometers and nanometers, respectively.

The term “about” when referring to a number means±5%. For example, the phrase “about 50 wt %” refers to a number between and including 47.500 and 52.500.

The phrase “nail coating composition” refers to a lacquer, a gel, or any other fluid, that is suitable to be applied to fingernails or toenails to decorate or protect the nail plates, that when hardened is a nail coating.

The phrase “nail coating,” refers to the hardened, fully cured substance covering a part or all of the nail, and any portions of this substance that extends or is built beyond the free edge of the nail. The phrase “nail coating” refers to finish product (which may then be buffed or filed), and may be composed of a single layer or multiple layers. The phrase “nail coating” is interpreted broadly, and it includes any hardened substance on a nail due to the application of a nail gel, a nail polish, a nail enamel or a nail varnish.

The terms “nail”, refer to either a fingernail or a toenail, either a natural nail or artificial nail. The term “nail” also refers to a human nail, as well as to any toughened keratin at the end of a digit of a non-human animal. As is understood from the context, the term “nail” may refer to a nail that has no coating on it, or a nail that already has several layers of a nail lacquer composition on it, or a nail that comprises a nail coating. Thus, the phrase “applying a layer of an actinic light curable dip gel onto a nail” means applying the gel onto a nail that has no coating on it, or that already has one or more layers of a nail lacquer on it.

The term “layer” refers to a single application of a nail coating composition onto a nail. As the context dictates, the term “layer” refers to an uncured layer, or a wet layer, of the nail coating composition, or the term refers to a dried layer, or partially dried or partially cured layer of the nail coating composition.

The term “client” refers to a person whose nails are being treated.

The phrase “nail technician” or “technician” is a worker skilled or licensed in the art of providing nail extensions, artificial nails, acrylic nails, gel nails, and other manicure services for clients. Alternative names for a nail technician may include a manicurist, or a cosmetologist. Such a person may work for pay at a nail salon or may be a manicure aficionado.

Under one embodiment of the present invention, the client and the nail technician are two different individuals. Although the description of the invention below describes the nail technician and the client as two separate individuals, it is understood that the claimed invention and methods are also suitable for use by a single person who is both a nail technician and a client. Under such embodiment of the present invention, the client and the nail technician are the same person.

The definition of the term “gel” and “dip gel” depends on the usage. The term “gel”, as it appears in the phrases such as “actinic light curable dip gel”, is defined broadly, and not restricted to any particular narrow definition. A gel is a fluid or a semi-fluid or jelly-like substance; it may be a Newtonian fluid, a rheopectic fluid, or thixotropic fluid. Further, the term “gel” and the phrase “dip gel”, as they appear in dependent claims or in paragraphs discussing actinic light curable dip gel, is used as a shorthand for the “actinic light curable dip gel”. The term “film-forming” and the phrase “film forming” are synomomous.

The term “acrylic” in the phrase “acrylic nail” refers to hardened polymerized composition used in manicure arts, which are composed of any of several types of poly ((meth)acrylates), or copolymers of various (meth)acrylate monomers, oligomers or copolymers of various (meth)acrylate monomers with any of several non-(meth)acrylic monomers.

When referring to a composition, the definition of the term “acrylate” as referred to in the monomeric form, includes an ester, a salt, or a conjugate base of acrylic acid, with the formula CH₂═CH—COO⁻. The definition of the term “acrylate” referred to in the polymeric or oligomeric form includes the repeating unit of an ester, a salt, or a conjugate base of acrylic acid, with the formula —[CH₂—CH(COO⁻)]—.

The definition of the term “methacrylate” as referred to in the monomeric form includes an ester, a salt, or a conjugate base of methacrylic acid, with the formula CH₂═C(CH₃)—COO⁻. The definition of the term “methacrylate” as referred to in the polymeric or oligomeric form includes an ester, a salt, or a conjugate base of methacrylic acid, with the formula —[CH₂═C(CH₃)—COO⁻]—.

The term “(meth)acrylate” means acrylate, methacrylate, or a mixture thereof. When referring to a compound, “(meth)acrylate” means an ester, a salt, or a conjugate base of an acrylic acid, with the formula CH₂═C(R)—COO⁻, wherein R is H, Me, or a mixture thereof. The definition of the term “(meth)acrylate” as referred to in the polymeric or oligomeric form includes an ester, a salt, or a conjugate base of methacrylic acid, with the formula —[CH₂═C(R)—COO⁻]—, wherein R is H, Me, or a mixture thereof. By extension, a monomer, oligomer, or polymer name containing as a part of its term the string “(meth)acrylate” should be interpreted as referring to the same monomer, oligomer, or polymer, that is an acrylate, methacrylate, or a mixture thereof. For example, the term “poly(C₁₋₁₂alkyl (meth)acrylate)” means “any of poly(C₁₋₁₂alkyl acrylate), poly(C₁₋₁₂alkyl methacrylate), and a mixture of poly(C₁₋₁₂alkyl acrylate) and poly(C₁₋₁₂alkyl methacrylate)”.

The term “polymer”, as for example in the phrase “polymer powder”, is to be interpreted broadly. Examples of polymers include both a homopolymer (a polymer comprising a single species of monomer) and a copolymer.

The term “copolymer” means a polymer comprising more than one species of monomer. The copolymer of the present invention consists of, or comprises essentially of, one or more linear chain copolymers. Examples of a copolymer include a statistical copolymer, a random copolymer, an alternating copolymer, a periodic copolymer, and a block copolymer. Under one embodiment of the present invention, the copolymer is a statistical copolymer or a random copolymer. The term “terpolymer” is a polymer comprising three species of monomer.

The phrase “alkyl group” relates to a linear or branched saturated hydrocarbon group, which is bound to the rest of the molecule by means of a single bond. The alkyl group may contain any number of carbons that would be appropriate for use in nail polish composition. The term “alkyl group”, unless specifically referred to otherwise, may be a branched alkyl group, a linear alkyl group. The adjective form “alkyl” without a noun that it modifies following it means an alkyl group; likewise, the term “methyl” without a noun that it modifies following it means a methyl group; etc.

The present invention relates to an actinic light curable dip gel. This actinic light curable dip gel is designed to be used with a powder to generate a nail coating. The nail coating may also comprise a base coat or a primer, and a top coat.

The actinic light curable dip gel of the present invention is a fully formulated composition, meaning that gel is designed to be applied to the client's nails without any further addition of ingredients.

The actinic light curable dip gel comprises a film forming resin; a monomer; a photoinitiator, and optionally, a solvent.

More specifically, the present invention is directed to an actinic light curable dip gel comprising: a film forming resin selected from the group consisting of poly(C₁₋₁₂alkyl(meth)acrylate) polymer, an acrylate functional acrylic resin, poly(meth)acrylate oligomer, styrene polymer, styrene divinylbenzene polymer, urethane polymer, cellulose acetate butyrate, cellulose acetate propionate, nitrocellulose, and mixtures thereof; a (meth)acrylate monomer; and one or more photoinitiators.

Under one embodiment, the present invention is directed to an actinic light curable dip gel comprising: a film forming acrylic resin selected from the group consisting of a poly(C₁₋₁₂alkyl(meth)acrylate) polymer, an acrylate functional acrylic resin, and mixtures thereof; and a monomer.

Examples of an acrylic resin include a poly(alkyl(meth)acrylate) polymer, wherein the alkyl groups comprise one to twelve carbon atoms, an acrylate functional acrylic resin, and mixtures thereof.

Suitable poly(alkyl(meth)acrylate) polymer is a poly(C₁₋₁₂alkyl (meth)acrylate) polymer. The poly(C₁₋₁₂alkyl (meth)acrylate) polymer consists of, or comprises mostly of, a polymer with repeating units —[CH₂═CR₁—COOR₂]—, wherein R₁ is either H or Me, and R₂ is an alkyl group with 1 to 12 carbons.

Under one embodiment the poly(C₁₋₁₂alkyl (meth)acrylate) polymer is a polymer prepared from one type of a monomer. Under this embodiment, the polymer consists of a single type of a polymer.

Under another embodiment the poly(C₁₋₁₂alkyl (meth)acrylate) polymer is a polymer prepared from more than one type of a monomer. Thus, a polymer of the present invention may have repeating units, wherein moieties of R₁ and R₂ of any one unit may not necessarily be the same as R₁ and R₂ of another unit. Under this embodiment the polymer may be considered a copolymer.

Under yet another embodiment, the phrase “poly(C₁₋₁₂alkyl (meth)acrylate) polymer” is a mixture of polymer, or mixture of copolymers.

Under alternative embodiments, the polymer consists of a mixture of poly(C₁₋₁₂alkyl acrylate) polymers; a mixture of poly(C₁₋₁₂alkyl methacrylate) polymers; a mixture of poly(C₁₋₁₂alkyl acrylate) polymer and poly(C₁₋₁₂alkyl methacrylate) polymer; a mixture of poly(C₁₋₁₂alkyl methacrylate) polymers and poly(C₁₋₁₂alkyl acrylate) polymer; or a mixture of poly(C₁₋₁₂alkyl methacrylate) polymers and poly(C₁₋₁₂alkyl methacrylate) polymer.

When mixed with a solvent, the polymer typically forms a homogenous suspension of the polymer in the composition. Alternatively, the polymer is already premixed with the solvent before the actinic light curable dip gel of the present invention is formulated.

Examples of poly(C₁₋₁₂alkyl (meth)acrylate) include poly(methyl (meth)acrylate), poly(ethyl (meth)acrylate), poly(propyl (meth)acrylate), poly(n-propyl (meth)acrylate), poly(isopropyl (meth)acrylate), poly(butyl (meth)acrylate), poly(n-butyl (meth)acrylate), poly(isobutyl (meth)acrylate), poly(sec-butyl (meth)acrylate), poly(pentyl (meth)acrylate), poly(hexyl (meth)acrylate), poly(heptyl (meth)acrylate), poly(octyl (meth)acrylate), poly(nonyl (meth)acrylate), poly(decyl (meth)acrylate), poly(undecyl (meth)acrylate), and poly(dodecyl (meth)acrylate). In these examples, the alkyl groups hexyl, heptyl, octyl, nonyl, decyl, undecyl, and dodecyl may be straight chain groups or branched groups.

Examples of a branched alkyl group include: 1-methylpropyl; sec-butyl; 2-methylpropyl; iso-butyl; 1,1-dimethylethyl; tert-butyl; 1-methylbutyl; sec-pentyl; 2-methylbutyl; 3-methylbutyl; 1-ethylpropyl; 3-pentyl; 1,1-dimethylpropyl; tert-pentyl; 1,2-dimethylpropyl; 2,2-dimethylpropyl; neopentyl; 1-methylpentyl; 2-methylpentyl; 3-methylpentyl; 4-methylpentyl; iso-amyl; 1,1-dimethylbutyl; 1,2-dimethylbutyl; 1,3-dimethylbutyl; 2,2-dimethylbutyl; 2,3-dimethylbutyl, 3,3-dimethylbutyl; 1-ethylbutyl; 2-ethylbutyl; 1,1,2-trimethylpropyl; 1,2,2-trimethylpropyl; 1-ethyl-1-methylpropyl, 1-ethyl-2-methylpropyl; 1-methylhexyl; 2-methylhexyl; 3-methylhexyl; 4-methylhexyl; 5-methylhexyl; 1,1-dimethylpentyl; 1,2-dimethylpentyl; 1,3-dimethylpentyl; 1,4-dimethylpentyl; 2,2-dimethylpentyl; 2,3-dimethylpentyl; 2,4-dimethylpentyl; 3,3-dimethylpentyl; 3,4-dimethylpentyl; 4,4-dimethylpentyl; 1-ethylpentyl; 2-ethylpentyl; 3-ethylpentyl; 1,1,2-trimethylbutyl; 1,1,3-trimethylbutyl; 1,2,2-trimethylbutyl; 1,2,3-trimethylbutyl; 1,3,3-trimethylbutyl; 2,2,3-trimethylbutyl; 2,3,3-trimethylbutyl; 1-(methylethyl)butyl; 1-ethyl-1-methylbutyl; 1-ethyl-3-methylbutyl; 2-(methylethyl)butyl; 2-ethyl-1-methylbutyl; 2-ethyl-2-methylbutyl; 2-ethyl-3-methylbutyl; 1-propylbutyl; 2-propylbutyl; 1,1,2,2-tetramethylpropyl; 1-ethyl-1,2-dimethylpropyl; 1-ethyl-2,2-dimethylpropyl; 1-ethyl-1,2-dimethylpropyl; 1-methylheptyl; 2-methylheptyl; 3-methylheptyl; 4-methylheptyl; 5-methylheptyl; 6-methylheptyl; 1,1-dimethylhexyl; 1,2-dimethylhexyl; 1,3-dimethylhexyl; 1,4-dimethylhexyl; 1,5-dimethylhexyl; 2,2-dimethylhexyl; 2,3-dimethylhexyl; 2,4-dimethylhexyl; 2,5-dimethylhexyl; 3,3-dimethylhexyl; 3,4-dimethylhexyl; 3,5-dimethylhexyl; 4,4-dimethylhexyl; 4,5-dimethylhexyl; 5,5-dimethylhexyl; 1-ethylhexyl; 2-ethylhexyl; 3-ethylhexyl; 4-ethylhexyl; 1,1,2-trimethylpentyl; 1,1,3-trimethylpentyl; 1,1,4-trimethylpentyl; 1,2,2-trimethylpentyl; 1,2,3-trimethylpentyl; 1,2,4-trimethylpentyl; 1,3,3-trimethylpentyl; 1,3,4-trimethylpentyl; 1,4,4-trimethylpentyl; 2,2,3-trimethylpentyl; 2,2,4-trimethylpentyl; 2,3,3-trimethylpentyl; 2,3,4-trimethylpentyl; 2,4,4-trimethylpentyl; 3,3,4-trimethylpentyl; 3,4,4-trimethylpentyl; 1-ethyl-1-methylpentyl; 1-ethyl-2-methylpentyl; 1-ethyl-3-methylpentyl; 1-ethyl-4-methylpentyl; 2-ethyl-1-methylpentyl; 2-ethyl-2-methylpentyl; 2-ethyl-3-methylpentyl; 2-ethyl-4-methylpentyl; 3-ethyl-1-methylpentyl; 3-ethyl-2-methylpentyl; 3-ethyl-3-methylpentyl; 3-ethyl-4-methylpentyl; 1-propylpentyl; 2-propylpentyl; 1-(methylethyl)pentyl; 2-(methylethyl)pentyl; 3-(methylethyl)pentyl; 1,1,2,2-tetramethylbutyl; 1,1,2,3-tetramethylbutyl; 1,1,3,3-tetramethylbutyl; 1,2,2,3-tetramethylbutyl; 1,2,3,3-tetramethylbutyl; 2,2,3,3-tetramethylbutyl; 1-ethyl-1,2-dimethylbutyl; 1-ethyl-1,3-dimethylbutyl; 1-ethyl-2,2-dimethylbutyl; 1-ethyl-2,3-dimethylbutyl; 1-ethyl-3,3-dimethylbutyl; 2-ethyl-1,1-dimethylbutyl; 2-ethyl-1,2-dimethylbutyl; 2-ethyl-1,3-dimethylbutyl; 2-ethyl-2,3-dimethylbutyl; 2-ethyl-3,3-dimethylbutyl; 1,1-diethylbutyl; 1,2-diethylbutyl; 2,2-diethylbutyl; 1-methyl-1-propylbutyl; 2-methyl-1-propylbutyl; 3-methyl-1-propylbutyl; 1-methyl-1-(methylethyl)butyl; 2-methyl-1-(methylethyl)butyl; 3-methyl-1-(methylethyl)butyl; 1-methyl-2-(methylethyl)butyl; 2-methyl-2-(methylethyl)butyl; 3-methyl-2-(methylethyl)butyl; 1-(1,1-dimethylethyl)butyl; mixtures thereof; and like. Further examples of branched alkyl groups include 8-methylnonyl, and 3,5,5-trimethylhexyl.

Although the poly(methyl(meth)acrylate) polymer may be present in a mixture of poly(C₁₋₁₂alkyl (meth)acrylate) polymers, the use of poly(methyl(meth)acrylate) polymer in a fully formulated nail coating composition formulation without any other poly(C₁₋₁₂alkyl (meth)acrylate) polymers typically yields a coating that is too hard for the powder particles to stick. To obtain soft nail coating, poly(methyl(meth)acrylate) polymer must either be mixed with polymers containing longer groups, or be augmented with plasticizers, or a combination thereof.

Under one embodiment, the poly(C₁₋₁₂alkyl (meth)acrylate) polymer is a poly(C₂₋₁₂alkyl (meth)acrylate.

Under one embodiment of the present invention, poly(C₁₋₁₂alkyl (meth)acrylate) polymer comprises a poly(C₁₋₄alkyl (meth)acrylate) polymer. Poly(C₁₋₄alkyl (meth)acrylate) is a polymer comprising —[CH₂═CR₁—COOR₂]— repeating units, wherein R₁ is either H or Me, and R₂ is an alkyl group with one to four carbons. Examples of poly(C₁₋₄alkyl (meth)acrylate) include poly(methyl (meth)acrylate), poly(methyl acrylate), poly(methyl methacrylate), poly(ethyl (meth)acrylate), poly(ethyl acrylate), poly(ethyl methacrylate), poly(propyl (meth)acrylate), poly(propyl acrylate), poly(propyl methacrylate), poly(butyl (meth)acrylate), poly(butyl acrylate), and poly(butyl methacrylate)

Further, the definition of poly(C₁₋₄alkyl (meth)acrylate) also includes the copolymers of poly(C₁₋₄alkyl (meth)acrylate). Under this embodiment, the poly(C₁₋₄alkyl (meth)acrylate) polymer is a copolymer prepared from one type of a monomer.

Under another embodiment, the poly(C₁₋₄alkyl (meth)acrylate) polymer is a polymer prepared from more than one type of a monomer. Thus, a polymer of the present invention may have repeating units —[CH₂═CR₁—COOR₂]—, wherein moieties of R₁ and R₂ of any one unit may not necessarily be the same as R₁ and R₂ of another unit.

Under yet another embodiment, the phrase “poly(C₁₋₄alkyl (meth)acrylate) polymer” is a mixture of polymer, or mixture of copolymers.

The above poly(C₁₋₁₂alkyl (meth)acrylate) polymer or the poly(C₁₋₄alkyl (meth)acrylate) polymer may be obtained from any number of commercial sources, including Dow (as PARALOID™), Evonik (as Degalan®), and like.

An acrylate functional acrylic resin is an acrylic resin obtained by reacting an acrylic acid with a poly(C₁₋₁₂alkyl (meth)acrylate) polymer, poly(C₁₋₁₂alkyl (meth)acrylate) copolymer, or poly(C₁₋₁₂alkyl (meth)acrylate)/styrene copolymer.

Other film forming resins which are suitable for the preparation of the actinic light curable dip gel include poly(meth)acrylate oligomer, styrene polymer, styrene divinylbenzene polymer, urethane polymer, cellulose acetate butyrate, cellulose acetate propionate, nitrocellulose, and mixtures thereof.

The phrase “poly(meth)acrylate oligomer” refers to the type of poly(meth)acrylate oligomers that are typically used in the personal care industry. Such oligomers have a functionality of about 2 to about 30. Examples of (meth)acrylate oligomers include urethane (meth)acrylate, epoxy (meth)acrylate, epoxy urethane (meth)acrylate, (meth)acrylated acrylate, (meth)acrylated polyether, (meth)acrylated polycarbonate, (meth)acrylated cellulose, (meth)acrylated butadiene, (meth)acrylated styrene, polyester (meth)acrylate, polyester urethane (meth)acrylate, polyether urethane (meth)acrylate, polybutadiene urethane (meth)acrylate, and a mixture thereof.

Styrene polymer, styrene divinylbenzene polymer, and urethane polymer are polymers and copolymers commonly used in nail care industry which are useful in preparing film formers. Under one embodiment, the styrene polymer is a copolymer of styrene with poly(meth)acrylate. Under one embodiment, the styrene divinylbenzene polymer is a copolymer of styrene divinylbenzene with poly(meth)acrylate. Under one embodiment, the urethane polymer is a copolymer of polyurethane with poly(meth)acrylate.

Nitrocellulose provides an unusual combination of properties of toughness, durability, solubility and solvent release, and is one of the preferred film-forming polymers. Examples of nitrocellulose are the so called nitrocellulose RS ⅛ sec. and ¼ sec.; nitrocellulose RS ½ sec.; and nitrocellulose RS 5-6 sec. and 60-80 sec. The term “RS” refers to the class of nitrocellulose with a nitrogen content of about 11.2 to about 12.8% with solubility in esters, ketones and glycol ethers manufactured by Dow (Wilmington, Del., US). The terms ⅛ sec., sec., ¼ sec., 5-6 sec., etc. represent viscosity and refer to the time it takes for a ball to fall to a given depth in the material. Other useful nitrocelluloses include A-grade nitrocellulose (nitrogen content 10.7% to 11.3%), AM-grade nitrocellulose (nitrogen content 11.3% to 11.8%), and E-grade nitrocellulose (nitrogen content 11.8% to 12.3%). Nitrocellulose is typically supplied in concentrations between 40% to 70% concentrations, wet with an alcohol or acetate solvent.

Under an embodiment, the actinic light curable dip gel comprises between 0 wt % and about 50 wt % of a poly(C₁₋₁₂alkyl(meth)acrylate) polymer. No more than about half of the actinic light curable dip gel is a poly(C₁₋₁₂alkyl(meth)acrylate) polymer.

The wt % is measured in reference to the entire actinic light curable dip gel, including the film former, monomer, photoinitiator, and any optional component, that is considered as a commercially viable product.

Under one embodiment, the balance of the wt % is the combination of a (meth)acrylate monomer, a photoinitiator, and optionally, an excipient such as a solvent. Under another embodiment, the balance of the wt % is the combination of a film forming resin selected from the group consisting of an acrylate functional acrylic resin, poly(meth)acrylate oligomer, styrene polymer, styrene divinylbenzene polymer, urethane polymer, cellulose acetate butyrate, cellulose acetate propionate, nitrocellulose, and mixtures thereof; a (meth)acrylate monomer, a photoinitiator, and optionally, an excipient such as a solvent.

Under an embodiment, the actinic light curable dip gel comprises between 0 wt % and about 50 wt % of an acrylate functional acrylic resin. No more than about half of the actinic light curable dip gel is an acrylate functional acrylic resin. Under one embodiment, the balance of the wt % is the combination of a (meth)acrylate monomer, a photoinitiator, and optionally, an excipient such as a solvent. Under another embodiment, the balance of the wt % is the combination of a film forming resin selected from the group consisting of poly(C₁₋₁₂alkyl(meth)acrylate) polymer, poly(meth)acrylate oligomer, styrene polymer, styrene divinylbenzene polymer, urethane polymer, cellulose acetate butyrate, cellulose acetate propionate, nitrocellulose, and mixtures thereof; a (meth)acrylate monomer, a photoinitiator, and optionally, an excipient such as a solvent.

Under an embodiment, the actinic light curable dip gel comprises between 0 wt % and about 50 wt % of a poly(meth)acrylate oligomer. No more than about half of the actinic light curable dip gel is a poly(meth)acrylate oligomer. Under one embodiment, the balance of the wt % is the combination of a (meth)acrylate monomer, a photoinitiator, and optionally, an excipient such as a solvent. Under another embodiment, the balance of the wt % is the combination of a film forming resin selected from the group consisting of poly(C₁₋₁₂alkyl(meth)acrylate) polymer, an acrylate functional acrylic resin, styrene polymer, styrene divinylbenzene polymer, urethane polymer, cellulose acetate butyrate, cellulose acetate propionate, nitrocellulose, and mixtures thereof; a (meth)acrylate monomer, a photoinitiator, and optionally, an excipient such as a solvent.

A combination of any of the embodiments above is possible. For example, under an embodiment, the actinic light curable dip gel comprises between 0 wt % and about 50 wt % of a poly(C₁₋₁₂alkyl(meth)acrylate) polymer, and between 0 wt % and about 50 wt % of an acrylate functional acrylic resin. Under one embodiment, the balance of the wt % is the combination of a (meth)acrylate monomer, a photoinitiator, and optionally, an excipient such as a solvent. Under another embodiment, the balance of the wt % is the combination of a film forming resin selected from the group consisting of a poly(meth)acrylate oligomer, styrene polymer, styrene divinylbenzene polymer, urethane polymer, cellulose acetate butyrate, cellulose acetate propionate, nitrocellulose, and mixtures thereof; a (meth)acrylate monomer, a photoinitiator, and optionally, an excipient such as a solvent.

Under an embodiment the actinic light curable dip gel comprises between 0 wt % and about 50 wt % of a poly(C₁₋₁₂alkyl(meth)acrylate) polymer, and between 0 wt % and about 50 wt % of a poly(meth)acrylate oligomer. Under one embodiment, the balance of the wt % is the combination of a (meth)acrylate monomer, a photoinitiator, and optionally, an excipient such as a solvent. Under another embodiment, the balance of the wt % is the combination of a film forming resin selected from the group consisting of an acrylate functional acrylic resin, styrene polymer, styrene divinylbenzene polymer, urethane polymer, cellulose acetate butyrate, cellulose acetate propionate, nitrocellulose, and mixtures thereof; a (meth)acrylate monomer, a photoinitiator, and optionally, an excipient such as a solvent.

Under an embodiment, the actinic light curable dip gel comprises between 0 wt % and about 50 wt % of an acrylate functional acrylic resin, and between 0 wt % and about 50 wt % of a poly(meth)acrylate oligomer. Under one embodiment, the balance of the wt % is the combination of a (meth)acrylate monomer, a photoinitiator, and optionally, an excipient such as a solvent. Under another embodiment, the balance of the wt % is the combination of a film-forming resin selected from the group consisting of poly(C₁₋₁₂alkyl(meth)acrylate) polymer, styrene polymer, styrene divinylbenzene polymer, urethane polymer, cellulose acetate butyrate, cellulose acetate propionate, nitrocellulose, and mixtures thereof; a (meth)acrylate monomer, a photoinitiator, and optionally, an excipient such as a solvent.

Under an embodiment, the actinic light curable dip gel comprises between 0 wt % and about 50 wt % of a poly(C₁₋₁₂alkyl(meth)acrylate) polymer, between 0 wt % and about 50 wt % of an acrylate functional acrylic resin, and between 0 wt % and about 50 wt % of a poly(meth)acrylate oligomer. Under one embodiment, the balance of the wt % is the combination of a (meth)acrylate monomer, a photoinitiator, and optionally, an excipient such as a solvent. Under another embodiment, the balance of the wt % is the combination of a film-forming resin selected from the group consisting of styrene polymer, styrene divinylbenzene polymer, urethane polymer, cellulose acetate butyrate, cellulose acetate propionate, nitrocellulose, and mixtures thereof; a (meth)acrylate monomer, a photoinitiator, and optionally, an excipient such as a solvent.

The actinic light curable dip gel of the present invention comprises one or more monomers. An example of such monomers includes (meth)acrylate monomers.

The (meth)acrylate monomer used in the actinic light curable dip gel may be any acrylate monomer or methacrylate monomer that is used in nail art formulations in which the curing is performed by actinic light. The (meth)acrylate monomer has a formula CH₂═C(R)—COOR′, wherein R is H, Me, or a mixture thereof, and R′ is an organic group. Examples of organic group R′ include hydrocarbons, that is, aliphatic (e.g., alkyl or alkenyl), alicyclic (e.g., cycloalkyl, cycloalkenyl) compounds, and aromatic-, aliphatic-, and alicyclic-substituted aromatic compounds, as well as cyclic compounds wherein the ring is completed through another portion of the molecule (e.g., two substituents together form an alicyclic compound); groups that include hetero atoms, that is, groups that contain other than carbon in a ring or chain otherwise composed of carbon atoms, such as oxygen, nitrogen, such as groups containing non-hydrocarbon groups, such as alkoxy, amino, amido, and similar groups.

Examples of (meth)acrylate include methyl acrylate, methyl methacrylate, ethyl acrylate, ethyl methacrylate, hydroxypropyl acrylate, hydroxypropyl methacrylate, butylacrylate, butyl methacrylate, hydroxyethyl acrylate, propyl methacrylate, isobutyl methacrylate, sec-butyl methacrylate, hydroxyethyl methacrylate, butoxyethyl acrylate, butoxyethyl methacrylate, diethylaminoethyl acrylate, diethylaminoethyl methacrylate, 2-ethylhexyl acrylate, 2-ethylhexyl methacrylate, ethoxyethyl acrylate, ethoxyethyl methacrylate, t-butyl aminoethyl acrylate, t-butyl aminoethyl methacrylate, methoxyethylene glycolacrylate, methoxyethylene glycol methacrylate, phosphoethyl acrylate, phosphoethyl methacrylate, methoxy propyl acrylate, methoxy propyl methacrylate, phenoxyethylene glycol acrylate, tetrahydrofurfuryl methacrylate, phenoxyethylene glycol methacrylate, caprolactone methacrylate, methacroyloxyethyl maleate, 2-hydroxyethyl methacrylate/succinate, phthalic acid monoethyl methacrylate, phenoxypolyethylene glycol acrylate, phenoxypolyethylene glycol methacrylate, 2-hydroxy-3-phenoxypropyl acrylate, 2-hydroxy-3-phenoxypropyl methacrylate, isobornyl acrylate, isobornyl methacrylate, 3-chloro-2-hydroxypropyl acrylate, 3-chloro-2-hydroxypropyl methacrylate, and mixtures thereof.

Under one embodiment, the at least one or more (meth)acrylate monomers comprise a methacrylate selected from the group consisting of hydroxypropyl methacrylate, hydroxyethyl methacrylate, ethyl methacrylate, propyl methacrylate, butyl methacrylate, isobutyl methacrylate, sec-butyl methacrylate, tetrahydrofurfuryl methacrylate, caprolactone methacrylate, methacroyloxyethyl maleate, 2-hydroxyethyl methacrylate/succinate, phthalic acid monoethyl methacrylate, or a mixture thereof.

Under one embodiment of the present invention, the at least one of the one or more (meth)acrylate monomers comprises a hydroxyl-containing (meth)acrylate monomer.

The actinic light curable dip gel of the present invention is designed to be used with a powder to generate a nail coating. The mixture of the dip gel and the powder also comprises a photoinitiator. There are various ways of introducing the photoinitiator to the mixture. Under one embodiment, the actinic light curable dip gel comprises the photoinitiator. Under another embodiment, the powder comprises the photoinitiator. Under yet another embodiment, both the dip gel and the powder comprise the photoinitiator. Under still another embodiment, the photoinitiator is added to the mixture of the dip gel and the powder either neat or in a vehicle comprising the photoinitiator.

Under one embodiment, the actinic light curable dip gel of the present invention comprises one or more photoinitiators. As described below, the photoinitiator is selected so that it is activated by photons of the wavelength associated with actinic light of, for example, a UV light lamp. Preferably, the photoinitiator should be active at the wavelength of actinic light of actinic light lamps commonly found in nail salons.

Such photoinitiators may be selected from benzyl ketones, monomeric hydroxyl ketones, polymeric hydroxyl ketones, α-amino ketones, acyl phosphine oxides, metallocenes, benzophenone, camphor quinone, and benzophenone derivatives. Specific examples of photoinitiators include 1-hydroxy-cyclohexylphenylketone; benzophenone; camphor quinone, 2-benzyl-2-(dimethylamino)-1-(4-(4-morphorlinyl)phenyl)-1-butanone; 2,2-dimethoxy-2-phenyl acetophenone; 2-methyl-1-(4-methylthio)phenyl-2-(4-morphorlinyl)-1-propanone; 2,4,6-trimethylbenzoyldiphenyl-phosphine oxide; bis(2,4,6-trimethyl benzoyl)phenyl phosphine oxide; diphenyl-(2,4,6-trimethylbenzoyl) phosphine oxide; bis(2,6-dimethoxybenzoyl-2,4,4-trimethyl pentyl)phosphine oxide; 2-hydroxy-2-methyl-1-phenyl-propan-1-one; phenyl bis(2,4,6-trimethylbenzoyl) phosphine oxide; benzyl-dimethylketal; isopropylthioxanthone; bis(η⁵-2,4-cyclopentadien-1-yl)bis[2,6-difluoro-3-(1H-pyrrol-1-yl)phenyl]titanium), and mixtures of any of the foregoing.

Under one embodiment of the present invention, the photopolymerizable composition comprises a single chemical compound that exhibits a photoinitiating properties. Under an alternative embodiment, the photoinitiator is a mixture of photoinitiators.

The photoinitiator is present in the photopolymerizable composition at amounts sufficient to be effective in aiding curing of the photopolymerizable composition. Such amounts may be determined empirically. The photopolymerizable composition comprises up to about 10 wt % of one or more photoinitiators. Under one embodiment, the photopolymerizable composition comprises about 0.5 to about 5.0 wt % of one or more photoinitiators.

One of the advantages of including the photoinitiator in the dip gel is that the dip gel can be used in combination with any commonly used powder.

One of the advantages of including the photoinitiator in the powder is that dip gel ceteris paribus, has a longer shelf life.

The actinic light curable dip gel of the present invention also optionally comprises a solvent. Under one embodiment, the solvent is selected from the group consisting of C₁₋₄ alkyl acetate, C₁₋₃ alcohol, C₃₋₅ ketone, and a mixture thereof.

The solvent C₁₋₄ alkyl acetate is a composition that consists of, consists essentially of, or comprises mostly of, a compound of formula CH₃—C(O)—OR′, wherein R′ is a —CH₃, methyl, —CH₂CH₃, ethyl, —CH₂CH₂CH₃, n-propyl, —CH(CH₃)₂, i-propyl, —CH₂CH₂CH₂CH₃, n-butyl, —CH(CH₃)CH₂CH₃, sec-butyl, —CH₂CH(CH₃)₂, isobutyl, —C(CH₃)₃, tert-butyl, and mixtures thereof.

Under one embodiment, the solvent is selected from the group consisting of n-butyl acetate, ethyl acetate, and mixtures thereof.

The solvent C₁₋₃ alcohol is a composition that consists of, consists essentially of, or comprises mostly of a compound of formula R′OH, wherein R′ is a —CH₃, methyl, —CH₂CH₃, ethyl, —CH₂CH₂CH₃, n-propyl, —CH(CH₃)₂, i-propyl, and mixtures thereof.

The solvent C₃₋₅ ketone is a composition that consists of, consists essentially of, or comprises mostly of a compound of formula R′—C(O)—R″, wherein R′ is a —CH₃, or —CH₂CH₃; R″ is —CH₃, —CH₂CH₃, —CH₂CH₂CH₃, or —CH(CH₃)₂ when R′ is —CH₃; and R″ is —CH₃, or —CH₂CH₃, when R′ is —CH₂CH₃; and mixtures thereof.

Examples of solvents include methanol, ethanol, isopropanol, n-butanol, s-butanol, i-butanol, methanol, ethanol, propanol, isopropanol, acetone, methylethyl ketone, butanone, methyl propyl ketone, 2-pentanone, diethyl ketone, 3-pentanone, methyl isopropyl ketone, 3-methylbutan-2-one, and mixtures thereof.

The present invention is also directed to an actinic light curable dip gel comprising an acrylic resin selected from the group consisting of a poly(C₁₋₁₂alkyl(meth)acrylate) polymer, an acrylate functional acrylic resin, and mixtures thereof; a monomer; at least one photoinitiator; and a solvent.

The present invention is also directed to an actinic light curable dip gel comprising an acrylic resin selected from the group consisting of a poly(_{C1-12}alkyl(meth)acrylate) polymer, an acrylate functional acrylic resin, and mixtures thereof; a monomer; at least one photoinitiator; a solvent; and a crosslinker.

The present invention is also directed to an actinic light curable dip gel comprising an acrylic resin selected from the group consisting of a poly(C₁₋₁₂alkyl(meth)acrylate) polymer, an acrylate functional acrylic resin, and mixtures thereof; a monomer; at least one photoinitiator; a solvent; and a silane coupling agent.

The present invention is also directed to an actinic light curable dip gel comprising an acrylic resin selected from the group consisting of a poly(C₁₋₁₂alkyl(meth)acrylate) polymer, an acrylate functional acrylic resin, and mixtures thereof; a monomer; at least one photoinitiator; a solvent; a crosslinker; and a silane coupling agent.

The present invention is also directed to an actinic light curable dip gel comprising an acrylic resin selected from the group consisting of poly(C₁₋₁₂alkyl(meth)acrylate) polymer, an acrylate functional acrylic resin, poly(meth)acrylate oligomer, styrene polymer, styrene divinylbenzene polymer, urethane polymer, cellulose acetate butyrate, cellulose acetate propionate, nitrocellulose, and mixtures thereof; a (meth)acrylate monomer; and one or more photoinitiators; wherein the actinic light curable dip gel comprises less than 50 wt % poly(C₁₋₁₂alkyl(meth)acrylate) polymer.

The present invention is also directed to an actinic light curable dip gel comprising an acrylic resin selected from the group consisting of poly(C₁₋₁₂alkyl(meth)acrylate) polymer, an acrylate functional acrylic resin, poly(meth)acrylate oligomer, styrene polymer, styrene divinylbenzene polymer, urethane polymer, cellulose acetate butyrate, cellulose acetate propionate, nitrocellulose, and mixtures thereof; a (meth)acrylate monomer; and one or more photoinitiators; wherein the actinic light curable dip gel comprises less than 50 wt % the acrylate functional acrylic resin.

The present invention is also directed to an actinic light curable dip gel comprising an acrylic resin selected from the group consisting of poly(C₁₋₁₂alkyl(meth)acrylate) polymer, an acrylate functional acrylic resin, poly(meth)acrylate oligomer, styrene polymer, styrene divinylbenzene polymer, urethane polymer, cellulose acetate butyrate, cellulose acetate propionate, nitrocellulose, and mixtures thereof; a (meth)acrylate monomer; and one or more photoinitiators; wherein the actinic light curable dip gel comprises less than 50 wt % the poly(meth)acrylate oligomer.

The present invention is also directed to an actinic light curable dip gel comprising an acrylic resin selected from the group consisting of poly(C₁₋₁₂alkyl(meth)acrylate) polymer, an acrylate functional acrylic resin, poly(meth)acrylate oligomer, styrene polymer, styrene divinylbenzene polymer, urethane polymer, cellulose acetate butyrate, cellulose acetate propionate, nitrocellulose, and mixtures thereof; a (meth)acrylate monomer; and one or more photoinitiators; wherein the actinic light curable dip gel comprises less than 40 wt % poly(C₁₋₁₂alkyl(meth)acrylate) polymer.

The present invention is also directed to an actinic light curable dip gel comprising an acrylic resin selected from the group consisting of poly(C₁₋₁₂alkyl(meth)acrylate) polymer, an acrylate functional acrylic resin, poly(meth)acrylate oligomer, styrene polymer, styrene divinylbenzene polymer, urethane polymer, cellulose acetate butyrate, cellulose acetate propionate, nitrocellulose, and mixtures thereof; a (meth)acrylate monomer; and one or more photoinitiators; wherein the actinic light curable dip gel comprises less than 40 wt % the acrylate functional acrylic resin.

The present invention is also directed to an actinic light curable dip gel comprising an acrylic resin selected from the group consisting of poly(C₁₋₁₂alkyl(meth)acrylate) polymer, an acrylate functional acrylic resin, poly(meth)acrylate oligomer, styrene polymer, styrene divinylbenzene polymer, urethane polymer, cellulose acetate butyrate, cellulose acetate propionate, nitrocellulose, and mixtures thereof; a (meth)acrylate monomer; and one or more photoinitiators; wherein the actinic light curable dip gel comprises less than 40 wt % the poly(meth)acrylate oligomer.

The present invention is also directed to an actinic light curable dip gel comprising an acrylic resin selected from the group consisting of poly(C₁₋₁₂alkyl(meth)acrylate) polymer, an acrylate functional acrylic resin, poly(meth)acrylate oligomer, styrene polymer, styrene divinylbenzene polymer, urethane polymer, cellulose acetate butyrate, cellulose acetate propionate, nitrocellulose, and mixtures thereof; a (meth)acrylate monomer; and one or more photoinitiators; wherein the actinic light curable dip gel comprises less than 30 wt % poly(C₁₋₁₂alkyl(meth)acrylate) polymer.

The present invention is also directed to an actinic light curable dip gel comprising an acrylic resin selected from the group consisting of poly(C₁₋₁₂alkyl(meth)acrylate) polymer, an acrylate functional acrylic resin, poly(meth)acrylate oligomer, styrene polymer, styrene divinylbenzene polymer, urethane polymer, cellulose acetate butyrate, cellulose acetate propionate, nitrocellulose, and mixtures thereof; a (meth)acrylate monomer; and one or more photoinitiators; wherein the actinic light curable dip gel comprises less than 30 wt % the acrylate functional acrylic resin.

The present invention is also directed to an actinic light curable dip gel comprising an acrylic resin selected from the group consisting of poly(C₁₋₁₂alkyl(meth)acrylate) polymer, an acrylate functional acrylic resin, poly(meth)acrylate oligomer, styrene polymer, styrene divinylbenzene polymer, urethane polymer, cellulose acetate butyrate, cellulose acetate propionate, nitrocellulose, and mixtures thereof; a (meth)acrylate monomer; and one or more photoinitiators; wherein the actinic light curable dip gel comprises less than 30 wt % the poly(meth)acrylate oligomer.

The present invention is also directed to an actinic light curable dip gel comprising an acrylic resin selected from the group consisting of poly(C₁₋₁₂alkyl(meth)acrylate) polymer, an acrylate functional acrylic resin, poly(meth)acrylate oligomer, styrene polymer, styrene divinylbenzene polymer, urethane polymer, cellulose acetate butyrate, cellulose acetate propionate, nitrocellulose, and mixtures thereof; a (meth)acrylate monomer; and one or more photoinitiators; wherein the actinic light curable dip gel comprises less than 20 wt % poly(C₁₋₁₂alkyl(meth)acrylate) polymer.

The present invention is also directed to an actinic light curable dip gel comprising an acrylic resin selected from the group consisting of poly(C₁₋₁₂alkyl(meth)acrylate) polymer, an acrylate functional acrylic resin, poly(meth)acrylate oligomer, styrene polymer, styrene divinylbenzene polymer, urethane polymer, cellulose acetate butyrate, cellulose acetate propionate, nitrocellulose, and mixtures thereof; a (meth)acrylate monomer; and one or more photoinitiators; wherein the actinic light curable dip gel comprises less than 20 wt % the acrylate functional acrylic resin.

The present invention is also directed to an actinic light curable dip gel comprising an acrylic resin selected from the group consisting of poly(C₁₋₁₂alkyl(meth)acrylate) polymer, an acrylate functional acrylic resin, poly(meth)acrylate oligomer, styrene polymer, styrene divinylbenzene polymer, urethane polymer, cellulose acetate butyrate, cellulose acetate propionate, nitrocellulose, and mixtures thereof; a (meth)acrylate monomer; and one or more photoinitiators; wherein the actinic light curable dip gel comprises less than 20 wt % the poly(meth)acrylate oligomer.

The present invention is also directed to an actinic light curable dip gel comprising an acrylic resin selected from the group consisting of poly(C₁₋₁₂alkyl(meth)acrylate) polymer, an acrylate functional acrylic resin, poly(meth)acrylate oligomer, styrene polymer, styrene divinylbenzene polymer, urethane polymer, cellulose acetate butyrate, cellulose acetate propionate, nitrocellulose, and mixtures thereof; a (meth)acrylate monomer; and one or more photoinitiators; wherein the actinic light curable dip gel comprises less than 10 wt % poly(C₁₋₁₂alkyl(meth)acrylate) polymer.

The present invention is also directed to an actinic light curable dip gel comprising an acrylic resin selected from the group consisting of poly(C₁₋₁₂alkyl(meth)acrylate) polymer, an acrylate functional acrylic resin, poly(meth)acrylate oligomer, styrene polymer, styrene divinylbenzene polymer, urethane polymer, cellulose acetate butyrate, cellulose acetate propionate, nitrocellulose, and mixtures thereof; a (meth)acrylate monomer; and one or more photoinitiators; wherein the actinic light curable dip gel comprises less than 10 wt % the acrylate functional acrylic resin.

The present invention is also directed to an actinic light curable dip gel comprising an acrylic resin selected from the group consisting of poly(C₁₋₁₂alkyl(meth)acrylate) polymer, an acrylate functional acrylic resin, poly(meth)acrylate oligomer, styrene polymer, styrene divinylbenzene polymer, urethane polymer, cellulose acetate butyrate, cellulose acetate propionate, nitrocellulose, and mixtures thereof; a (meth)acrylate monomer; and one or more photoinitiators; wherein the actinic light curable dip gel comprises less than 10 wt % the poly(meth)acrylate oligomer.

The present invention is also directed to an actinic light curable dip gel comprising an acrylic resin selected from the group consisting of poly(C₁₋₁₂alkyl(meth)acrylate) polymer, an acrylate functional acrylic resin, poly(meth)acrylate oligomer, styrene polymer, styrene divinylbenzene polymer, urethane polymer, cellulose acetate butyrate, cellulose acetate propionate, nitrocellulose, and mixtures thereof; a (meth)acrylate monomer; and one or more photoinitiators; and a solvent.

The present invention is also directed to an actinic light curable dip gel comprising an acrylic resin selected from the group consisting of poly(C₁₋₁₂alkyl(meth)acrylate) polymer, an acrylate functional acrylic resin, poly(meth)acrylate oligomer, styrene polymer, styrene divinylbenzene polymer, urethane polymer, cellulose acetate butyrate, cellulose acetate propionate, nitrocellulose, and mixtures thereof; a (meth)acrylate monomer; and one or more photoinitiators; and a crosslinker.

The present invention is also directed to an actinic light curable dip gel comprising an acrylic resin selected from the group consisting of poly(C₁₋₁₂alkyl(meth)acrylate) polymer, an acrylate functional acrylic resin, poly(meth)acrylate oligomer, styrene polymer, styrene divinylbenzene polymer, urethane polymer, cellulose acetate butyrate, cellulose acetate propionate, nitrocellulose, and mixtures thereof; a (meth)acrylate monomer; and one or more photoinitiators; a solvent; and a crosslinker.

The present invention is also directed to an actinic light curable dip gel comprising an acrylic resin selected from the group consisting of a poly(C₁₋₁₂alkyl(meth)acrylate) polymer, an acrylate functional acrylic resin, and mixtures thereof; a monomer; at least one photoinitiator; a solvent; and a silane coupling agent, wherein the actinic light curable dip gel comprises less than 1 wt % oligomers.

The present invention is also directed to an actinic light curable dip gel comprising an acrylic resin selected from the group consisting of a poly(C₁₋₁₂alkyl(meth)acrylate) polymer, an acrylate functional acrylic resin, and mixtures thereof; a monomer; at least one photoinitiator; a solvent; a crosslinker; and a silane coupling agent, wherein the actinic light curable dip gel comprises less than 1 wt % oligomers.

The present invention is also directed to an actinic light curable dip gel comprising an acrylic resin selected from the group consisting of a poly(C₁₋₁₂alkyl(meth)acrylate) polymer, an acrylate functional acrylic resin, and mixtures thereof; a monomer; at least one photoinitiator; and a solvent, wherein the actinic light curable dip gel comprises less than 2 wt % oligomers.

The present invention is also directed to an actinic light curable dip gel comprising an acrylic resin selected from the group consisting of a poly(C₁₋₁₂alkyl(meth)acrylate) polymer, an acrylate functional acrylic resin, and mixtures thereof; a monomer; at least one photoinitiator; a solvent; and a crosslinker, wherein the actinic light curable dip gel comprises less than 2 wt % oligomers.

The present invention is also directed to an actinic light curable dip gel comprising an acrylic resin selected from the group consisting of a poly(C₁₋₁₂alkyl(meth)acrylate) polymer, an acrylate functional acrylic resin, and mixtures thereof; a monomer; at least one photoinitiator; a solvent; and a silane coupling agent, wherein the actinic light curable dip gel comprises less than 2 wt % oligomers.

The present invention is also directed to an actinic light curable dip gel comprising an acrylic resin selected from the group consisting of a poly(C₁₋₁₂alkyl(meth)acrylate) polymer, an acrylate functional acrylic resin, and mixtures thereof; a monomer; at least one photoinitiator; a solvent; a crosslinker; and a silane coupling agent, wherein the actinic light curable dip gel comprises less than 2 wt % oligomers.

The present invention is also directed to an actinic light curable dip gel comprising an acrylic resin selected from the group consisting of a poly(C₁₋₁₂alkyl(meth)acrylate) polymer, an acrylate functional acrylic resin, and mixtures thereof; a monomer; at least one photoinitiator; and a solvent, wherein the actinic light curable dip gel comprises less than 5 wt % oligomers.

The present invention is also directed to an actinic light curable dip gel comprising an acrylic resin selected from the group consisting of a poly(C₁₋₁₂alkyl(meth)acrylate) polymer, an acrylate functional acrylic resin, and mixtures thereof; a monomer; at least one photoinitiator; a solvent; and a crosslinker, wherein the actinic light curable dip gel comprises less than 5 wt % oligomers.

The present invention is also directed to an actinic light curable dip gel comprising an acrylic resin selected from the group consisting of a poly(C₁₋₁₂alkyl(meth)acrylate) polymer, an acrylate functional acrylic resin, and mixtures thereof; a monomer; at least one photoinitiator; a solvent; and a silane coupling agent, wherein the actinic light curable dip gel comprises less than 5 wt % oligomers.

The present invention is also directed to an actinic light curable dip gel comprising an acrylic resin selected from the group consisting of a poly(C₁₋₁₂alkyl(meth)acrylate) polymer, an acrylate functional acrylic resin, and mixtures thereof; a monomer; at least one photoinitiator; a solvent; a crosslinker; and a silane coupling agent, wherein the actinic light curable dip gel comprises less than 5 wt % oligomers.

Under one embodiment the actinic light curable dip gel further comprises a crosslinker. Under another embodiment the monomer liquid comprises two or more different compounds that are crosslinkers.

Generally, a cross-linker is a multifunctional monomer. Examples of crosslinkers include diacrylates, triacrylates, tetraacrylates, pentaacrylates and higher acrylates. Such examples include trimethylolpropane triacrylate, trimethylolethane triacrylate, trimethylolpropane trimethacrylate, trimethylolethane trimethacrylate, tetramethylene glycol dimethacrylate, triethylene glycol dimethacrylate, tetraethylene glycol diacrylate, pentaerythritol diacrylate, penta-erythritol triacrylate, pentaerythritol tetraacrylate, dipentaerythritol diacrylate, dipenta-erythritol triacrylate, dipentaerythritol tetraacrylate, dipentaerythritol pentaacrylate, dipenta-erythritol hexaacrylate, tripentaerythritol octaacrylate, pentaerythritol dimethacrylate, penta-erythritol trimethacrylate, dipentaerythritol dimethacrylate, dipentaerythritol tetramethacrylate, tripentaerythritol octamethacrylate, ethylene glycol diacrylate, ethylene glycol dimethacrylate, 1,3-butanediol diacrylate, 1,3-butanediol dimethacrylate, sorbitol triacrylate, sorbitol tetraacrylate, pentaerythritol-modified triacrylate, sorbitol tetramethacrylate, sorbitol pentaacrylate, sorbitol hexaacrylate, oligoester acrylates and methacrylates, glycerol di- and triacrylate, 1,4-cyclohexane diacrylate, bisacrylates and bismethacrylates of polyethylene glycol, and mixtures thereof.

Under one embodiment, at least one of the one or more crosslinker is selected from the group consisting of trimethylol propane tri(meth)acrylate, ethoxylated glycerin tri(meth)acrylate, ethoxylated trimethylolpropane tri(meth)acrylate, ditrimethylol propane tetra(meth)acrylate, pentaerythritol tri(meth)acrylate, pentaerythritol tetra(meth)acrylate, propoxylated pentaerythritol tetra(meth)acrylate, ethoxylated pentaerythritol tetra(meth)acrylate, dipentaerythritol hexa(meth)acrylate, dipentaerythritol penta(meth)acrylate and ethoxylated iscyanuric acid tri(meth)acrylates.

Under one embodiment the crosslinkers comprise methacrylate groups. Example of such crosslinkers include dimethacrylates, trimethacrylates, tetramethacrylate, and higher methacrylates. Examples of such methacrylic crosslinkers include trimethylolpropane trimethacrylate, trimethylolethane trimethacrylate, tetramethylene glycol dimethacrylate, triethylene glycol dimethacrylate, pentaerythritol dimethacrylate, penta-erythritol trimethacrylate, dipentaerythritol dimethacrylate, dipentaerythritol tetramethacrylate, tripentaerythritol octamethacrylate, 1,3-butanediol dimethacrylate, sorbitol tetramethacrylate, oligoester methacrylates, bismethacrylates of polyethylene glycol having a molecular weight of from 200 to 1500, and mixtures thereof. For example, trimethylolpropane trimethacrylate is a composition consisting of, or comprising largely of, the compound of formula (CH₂═CMe-C(O)—O—CH₂)₃C—C₂H₅. It is a low volatility trifunctional monomer offering fast cure response in free radical polymerization.

Another suitable crosslinker is an alkoxylated crosslinker, with the formula (CH₂═CMe-C(O)—O-(AO)ₓ—CH₂—)₃C—R; wherein wherein R is a C₁ to C₆ alkyl group; AO is a small alkoxy group, such as an ethylene oxide, —CH₂—CH₂—O—, propylene oxide, —CH(CH₃)—CH₂—O—, —CH₂—CH₂—CH₂—O—, butylene oxide, and —CH(Et)—CH₂—O—; and wherein for each (CH₂═CMe-C(O)—O-(AO)ₓ—CH₂—) group x is independently 0, 1, 2, or 3. Using R=ethyl, and AO=ethylene oxide as an example, an exemplary alkoxylated crosslinker has a structure of formula

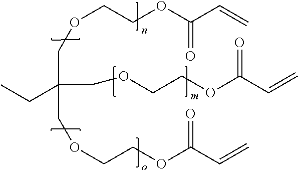

wherein m, n, and o are each independently 0, 1, 2, or 3.

Under one embodiment the actinic light curable dip gel further comprises a silane coupling agent.

The silane coupling agent is an organofunctional silane which comprises a reactive functional group.

The silane coupling agent acts as a surface modifier and which has suitable reactive functional groups.

Examples of the silane coupling agent includes 3-methacryloyloxypropyl-trimethoxysilane (Dynasylan MEMO, Silquest A-174NT), vinyltrimethoxysilane (Dynasylan VTMO or VTEO, Silquest A-151 or A-171), methyltri(m)ethoxysilane (Dynasylan MTMS or MTES), 3-mercaptopropyltrimethoxy-silane (Dynasylan MTMO; Silquest A-189), 3-glycidyloxy-propyltrimethoxysilane (Dynasylan GLYMO, Silquest A-187), tris[3-(trimethoxysilyl)propyl]isocyanurate (Silquest Y-11597), bis[3-(triethoxysilyl)propyl)]tetrasulfide (Silquest A-1289), bis[3-(triethoxy-silyl)propyl disulfide (Silquest A-1589, beta-(3,4-epoxycyclohexyl)ethyltrimethoxysilane (Silquest A-186), bis(triethoxysilyl)ethane (Silquest Y-9805), gamma-isocyanatopropyltrimethoxysilane (Silquest A-Link 35, GENIOSIL GF40), methacryloyloxymethyltri(m)ethoxysilane (GENIOSIL XL 33, XL 36), (methacryloyloxymethyl(m)-ethyldimethoxysilane (GENIOSIL XL 32, XL 34), (isocyanatomethyl)methyldimethoxysilane, (isocyanato-methyl)trimethoxysilane, 3-(triethoxysilyl)propyl-succinic anhydride (GENIOSIL GF 20), (methacryloyloxy-methyl)methyldiethoxysilane, 2-acryloyloxyethylmethyl-dimethoxysilane, 2-methacryloyloxyethyltrimethoxy-silane, 3-acryloyloxypropylmethyldimethoxysilane, 2-acryloyloxyethyltrimethoxysilane, 2-methacryloyloxy-ethyltriethoxysilane, 3-acryloyloxypropyltrimethoxy-silane, 3-acryloyloxypropyltripropoxysilane, 3-meth-acryloyloxypropyltriethoxysilane, 3-methacryloyloxy-propyltriacetoxysilane, 3-methacryloyloxypropylmethyl-dimethoxysilane, vinyltrichlorosilane, vinyltrimethoxy-silane (GENIOSIL XL 10), vinyltris(2-methoxyethoxy)silane (GENIOSIL GF 58), and vinyltriacetoxysilane.

Under one embodiment, the actinic light curable dip gel comprises an acrylic resin and a monomer, but very little or no oligomer.

Under one embodiment, the actinic light curable dip gel comprises a film forming acrylic resin selected from the group consisting of a poly(C₁₋₁₂alkyl(meth)acrylate) polymer, an acrylate functional acrylic resin, and mixtures thereof; and a monomer; wherein the actinic light curable dip gel comprises less than 0.5 wt % oligomers.

Under one embodiment, the actinic light curable dip gel comprises a film forming acrylic resin selected from the group consisting of a poly(C₁₋₁₂alkyl(meth)acrylate) polymer, an acrylate functional acrylic resin, and mixtures thereof; and a monomer; wherein the actinic light curable dip gel comprises less than 1.0 wt % oligomers.

Under one embodiment, the actinic light curable dip gel comprises a film forming acrylic resin selected from the group consisting of a poly(C₁₋₁₂alkyl(meth)acrylate) polymer, an acrylate functional acrylic resin, and mixtures thereof; and a monomer; wherein the actinic light curable dip gel comprises less than 5.0 wt % oligomers.

The actinic light curable dip gel optionally comprises additional ingredients, such a colorant or a pigment.

One purpose of using pigment in the composition of the present invention is to provide a tint or a color to the formed nail coating. The exhibition of such a tint or a color may provide a contrasting background onto which the powdered layer is applied. Under this embodiment, the contrast between the soft nail coating and the powder allows the technician to apply an even coat of powder. For example, an area of a nail has the hue of the soft nail coating may be deemed to have too little powder coating.

Another purpose of using a pigment is to provide a whitish appearance to the photopolymerizable composition, so that it appears as an attractive, clean product to the nail technician.

Examples of pigments may be incorporated into the composition of the present invention include: annatto, caramel, carmine, ß-carotene, potassium sodium copper chlorophyllin (chlorophyllin copper complex), dihydroxyacetone, bismuth oxychloride, guaiazulene, iron oxides, ferric ammonium ferrocyanide, ferric ferrocyanide, chromium hydroxide green, chromium oxide greens, guanine, pyrophyllite, mica, silver, titanium dioxide, aluminum powder, bronze powder, copper powder, ultramarines, manganese violet, zinc oxide, luminescent zinc sulfide, FD&C Blue No. 1, D&C Blue No. 4, Iron Blue, D&C Brown No. 1, FD&C Green No. 3, D&C Green No. 5, D&C Green No. 6, D&C Green No. 8, D&C Orange No. 4, D&C Orange No. 5, D&C Orange No. 10, D&C Orange No. 11, FD&C Red No. 4, D&C Red No. 6, D&C Red No. 7, D&C Red No. 17, D&C Red No. 21, D&C Red No. 22, D&C Red No. 27, D&C Red No. 28, D&C Red No. 30, D&C Red No. 31, D&C Red No. 33, D&C Red No. 34, D&C Red No. 36, FD&C Red No. 40, D&C Violet No. 2, Ext. D&C Violet No. 2, FD&C Yellow No. 5, FD&C Yellow No. 6, D&C Yellow No. 7, Ext. D&C Yellow No. 7, D&C Yellow No. 8, D&C Yellow No. 10, D&C Yellow No. 11, and mixture of any of the foregoing.

The present invention is also directed to a method of forming a nail cover comprising the steps of applying a layer of the actinic light curable dip gel of claim 1 onto a nail, applying a powder onto the nail, and exposing the nail to actinic light. These steps may be carried out in any order. Any of these steps may be repeated. Additional steps may also be a part of this method.

This general method may be exemplified by at least four methods of forming a nail cover.

Under one method of forming a nail cover, the method of forming a nail cover comprises the steps of (a)(i) applying a layer of the actinic light curable dip gel onto a nail; (ii) introducing a powder onto the nail; (iii) repeating steps (i) and (ii) 0 to 5 times; and (b) exposing the nail to actinic light.

In this method, the steps (a)(i) and (a)(ii) are repeated zero to five times, and then the nail is exposed to the actinic light.

Under one embodiment, steps (a)(i) and (a)(ii) are done only once. The method of forming a nail cover under this embodiment comprises the steps of applying a layer of the actinic light curable dip gel onto a nail; dipping the nail into a polymer powder; and exposing the nail to actinic light.

The invention solves at least one of the problems associated with traditional nail dip systems. Such problems include skill level needed to form a nail coating with the traditional nail dip system, and the length of time needed to form a nail coating with the traditional nail dip system.

One of the advantages of the present invention is that fewer coatings are necessary in order to result in a finished nail.

Under an alternative embodiment, steps (a)(i) and (a)(ii) are repeated once. The method of forming a nail cover under this embodiment comprises the steps of applying a layer of the actinic light curable dip gel onto a nail; applying a polymer powder onto the nail; applying a second layer of the actinic light curable dip gel onto a nail; applying the polymer powder onto the nail for the second time; and exposing the nail to actinic light.

Under an alternative embodiment, steps (a)(i) and (a)(ii) are repeated twice. The method of forming a nail cover under this embodiment comprises the steps of applying a layer of the actinic light curable dip gel of onto a nail; applying a polymer powder onto the nail; applying a second layer of the actinic light curable dip gel onto a nail; applying the polymer powder onto the nail for the second time; applying a third layer of the actinic light curable dip gel of onto a nail; applying the polymer powder onto the nail for the third time; and exposing the nail to actinic light.

Under an alternative embodiment, steps (a)(i) and (a)(ii) are repeated three times. The method of forming a nail cover under this embodiment comprises the steps of applying a layer of the actinic light curable dip gel of onto a nail; applying a polymer powder onto the nail; applying a second layer of the actinic light curable dip gel onto a nail; applying the polymer powder onto the nail for the second time; applying a third layer of the actinic light curable dip gel of onto a nail; applying the polymer powder onto the nail for a third time; applying a fourth layer of the actinic light curable dip gel of onto a nail; applying the polymer powder onto the nail for the fourth time; and exposing the nail to actinic light.

Under an alternative embodiment, steps (a)(i) and (a)(ii) are repeated four times. The method of forming a nail cover under this embodiment comprises the steps of applying a layer of the actinic light curable dip gel of onto a nail; applying a polymer powder onto the nail; applying a second layer of the actinic light curable dip gel onto a nail; applying the polymer powder onto the nail for the second time; applying a third layer of the actinic light curable dip gel of onto a nail; applying the polymer powder onto the nail for a third time; applying a fourth layer of the actinic light curable dip gel of onto a nail; applying the polymer powder onto the nail for the fourth time; applying a fifth layer of the actinic light curable dip gel of onto a nail; applying the polymer powder onto the nail for the fifth time; and exposing the nail to actinic light.

Under an alternative embodiment, steps (a)(i) and (a)(ii) are repeated five times. The method of forming a nail cover under this embodiment comprises the steps of applying a layer of the actinic light curable dip gel of onto a nail; applying a polymer powder onto the nail; applying a second layer of the actinic light curable dip gel onto a nail; applying the polymer powder onto the nail for the second time; applying a third layer of the actinic light curable dip gel of onto a nail; applying the polymer powder onto the nail for a third time; applying a fourth layer of the actinic light curable dip gel of onto a nail; applying the polymer powder onto the nail for the fourth time; applying a fifth layer of the actinic light curable dip gel of onto a nail; applying the polymer powder onto the nail for the fifth time; applying a sixth layer of the actinic light curable dip gel of onto a nail; applying the polymer powder onto the nail for the sixth time; and exposing the nail to actinic light.

In step (a)(i) of the method of forming a nail cover, the actinic light curable dip gel is applied onto the nail. This gel is applied by any of the conventional means available to the nail technician, including using a brush, a nail coating brush, a dapper, an orange stick, and like.

After the transfer of the actinic light curable dip gel onto the nail, the gel is spread out to make a layer of the actinic light curable dip gel. The spreading of the gel may be done by the same tool as was the performance of the transfer of the gel, or it may be done by another tool, or a combination of tools. Under one embodiment, at least some of the spreading may be done without any tools, as the gel may spread on the nail by itself.

The actinic light curable dip gel is brushed onto the nail, resulting in a layer of dip gel layer. The layer can be of any thickness desired to match the desired physical characteristic and effect of the formed nail coating.

Under one embodiment at least one of the layers formed by one application of the actinic light curable dip gel is thin. A typical thin layer has a thickness range of about 20 to about 120 micrometers. Using several thin layers, as opposed to fewer thicker layers, may generate a more uniform, a more durable coat. Moreover, using thinner layers interspersed with polymer powder may aid in a greater interaction of the polymer powder with the actinic light curable dip gel.

Under one embodiment at least one of the layers formed by one application of the actinic light curable dip gel is thick. A typical thick layer has a thickness of about 100 to 250 micrometers. Using fewer thicker layers may aid in increasing the speed at which the final nail coating is obtained.

Under one embodiment at least one of the layers formed by one application of the actinic light curable dip gel has an uneven thickness. Such unevenness may aid in forming a nail coating that exhibits a gradual change in a characteristic of the nail coating, such as a color gradient. An example of a color gradient is a nail covering which is lighter at the proximal side or the nail or the lunula, and is darker at the distal side of the nail or the free edge.

In step (a)(ii) of the method of forming a nail cover, the polymer powder is applied onto the nail. The nail is covered by a layer of the actinic light curable dip gel, and upon application of the polymer powder onto the nail, the polymer powder sticks to the gel. After the polymer powder sticks to the gel, the gel interacts with the polymer powder to create a uniform layer.

The application of the polymer powder onto the nail may be achieved in any method that exposes the polymer powder to the actinic light curable dip gel layer.

Under one embodiment the nail may be dipped into a vessel of the polymer powder. The vessel may be a jar or a bottle of the polymer powder in which the polymer powder is typically sold. The vessel may be a weighing boat, a bowl, a cup, a jar, or any other open container into which some amount of the polymer powder to transferred prior to the dipping of the nail.

Under one embodiment the whole finger is inserted into the vessel of the polymer powder. The polymer powder sticks substantially to the nail covered with a layer of actinic light curable dip gel, and very little of the polymer powder sticks to the rest of the finger.

Under another embodiment, only a portion of the finger is exposed to the polymer powder. For example, the finger may be positioned so that the nail is substantially parallel to the surface of a pile of the polymer powder, and contact is made with the surface.

Under still another embodiment, the nail technician sprinkles the polymer powder onto the nail.

The application of the polymer powder onto the nail occurs prior to curing of the actinic light curable dip gel. The period of time from the application of the actinic light curable dip gel to the beginning of the application of the polymer powder may be as little as 5 seconds, or as long as 10 minutes.

The best performance for applying the polymer powder to a nail is expected when the gel is wet (i.e., fluid). Because the actinic light curable dip gel is designed to be cured mostly by actinic light, the time for the application of the polymer powder may be as long as 15 minutes or an hour.

However, shorter period of time from the application of the actinic light curable dip gel to the beginning of the application of the polymer powder appears to have advantages over waiting for a longer time. One such advantage is that the nails may be done faster. A second advantage is that the nail coating exhibit a more intense color from the powder. A third advantage is that the nail coating exhibit greater opacity.

The period of time of dipping of the nail into a vessel of the polymer powder may be as short as 3 seconds. After about the three seconds in the polymer powder, the nail is removed from the vessel of the polymer powder. Longer periods of time of dipping of the nail do not negatively affect the quality of nail coating.

After the nail is removed from the vessel of polymer powder, the powder wets out. Once the powder wets out, any excess polymer powder is then gently tapped off or brushed off the nail. After this wet-out time, no polymer powder should remain on the cuticle or accumulated in the lateral nail fold.

Under one embodiment, the powder is similar to, or is the same as, the polymer powder that is typically used in the acrylic nail industry. When mixed with the actinic light curable dip gel, the actinic light curable dip gel is a continuous phase, and the polymer powder is a discontinuous phase.

Under one embodiment, the polymer powder comprises a polyalkyl(meth)acrylate, or a mixture thereof, wherein the alkyl groups comprise one to twelve carbon atoms. Examples of polyalkyl(meth)acrylates include poly(ethyl methacrylate), poly(methyl methacrylate), PEMA, PMMA, and copolymers thereof.

The phrase “polymer powder”, regardless of the polymer composition, refers to both the cross-linked polymer powder and to the polymer powder that is not crosslinked. Under one embodiment the polymer powder is crosslinked. Under another embodiment the polymer powder is not crosslinked.

The polymer powder is a powder which may be prepared by a routine technique, such as suspension polymerization in which the reaction takes place between droplets of the corresponding monomer suspended in a solution of water and catalyst. The molecular weight of the polymer suitable for use in the present invention is similar to the polymers used in the acrylic nail industry.

The polymer powder prior to mixing with other ingredients consists of fine microspheres. The particle size of these microspheres is the same or similar to the particle size of microspheres commonly used by nail salons. Under one embodiment of the present invention, the mean particle size is greater than about 100 μm. Under one embodiment of the present invention, the mean particle size is less than about 100 μm. Under another embodiment of the present invention, the mean particle size is between about 50 μm and about 100 μm. Under still another embodiment of the present invention, the mean particle size is between about 10 μm and about 50 μm. Under yet another embodiment of the present invention, the mean particle size is less than 10 μm.

The present invention is also directed to the use of the actinic light curable dip gel with powder, wherein the powder does not chemically react, or does not substantially chemically react, with the actinic light curable dip gel. The nail cover, once cured, is a solid comprising at least two distinct phases: a continuous phase, and a discontinuous phase. The continuous phase is cured dip gel. The discontinuous phase is the powder that did not chemically react, or did not substantially chemically react, with the actinic light curable dip gel.

The present invention is also directed to a method of forming a nail cover comprising the steps of (a)(i) applying a layer of the actinic light curable dip gel onto a nail; (ii) applying onto the nail a powder selected from the group consisting of: special effects pigments, spherical aliphatic polyurethane beads, micronized polyethylene and PTFE, micronized polypropylene wax, silicone resin powder, styrene polymer, styrene copolymer, glass beads, nylon beads and polyethylene terephthalate based effect pigments, and mixtures thereof; (iii) repeating steps (i) and (ii) 0 to 5 times; and (b) exposing the nail to actinic light.

The present invention is also directed to a method of forming a nail cover comprising the steps of (a) applying a layer of the actinic light curable dip gel onto a nail; (b) applying onto the nail a powder selected from the group consisting of: special effects pigment, spherical aliphatic polyurethane beads, micronized polyethylene and PTFE, micronized polypropylene wax, silicone resin powder, styrene polymer, styrene copolymer, glass beads, nylon beads and polyethylene terephthalate based effect pigments, and mixtures thereof; (c) exposing the nail to actinic light; and (d) repeating steps (a) to (c) 0 to 5 times.

The special effects pigment is selected from the group consisting of mica, aluminum, calcium borosilicate, titanium, and synthetic mica.

Under an alternative embodiment, the special effects pigment is comprised of a material selected from the group consisting of a metal, a metal oxide, a main group oxide, a metal/main group oxide, glitter, and a combination thereof.

The material from which the powder particles are made is nonreactive with the polymers that make up the soft bottom layer, nor with polymers which may be used as a top layer. Non-reactive means that there is no chemical reaction at all, or that there is no appreciable reaction.

Further, the material is non-toxic at the levels to the client and/or to the nail technician are exposed to powder when the powder is applied or when worn.

Examples of metals include transition metals and main group metals. Examples of suitable transition metals include titanium, chromium, iron, cobalt, nickel, palladium, platinum, copper, silver, gold, and zinc. Examples of main group metals include aluminum, tin, and bismuth. Suitable metals include coinage metals or precious metals. Further, particularly suited materials include silver and aluminum.

Additionally, metals also include alloys of any transition metal or a main group metal with other transition metals or main group metals.

Metal oxides include both transition metal oxides and main group metal oxides, such as tin oxide, iron oxide and aluminum oxides.

Metal oxides include main group metal oxides, main group non-metal oxides, and a combination thereof. Examples include silicon oxide, glass, aluminum oxide, tin oxide, calcium aluminum borosilicate, and bismuth oxychloride.

The term “glitter” means any material which may be used in the cosmetic industry, the particles of which reflect light in various angles, causing the surface comprising such material to sparkle or shimmer. Glitters are small flat reflective particles are typically prepared from thin plastic sheets coated with a reflective material. The composition of glitter includes a polymer and any of a metal, a metal oxide, a main group oxide, a metal/main group oxide, or a combination thereof.

Additional suitable materials from which powder particles are comprised of include a combination material of any of the preceding. Examples include phyllosilicate, synthetic fluorphlogopite, mica, and other minerals, such as biotite, lepidolite, phlogopite, muscovite, clintonite, and zinnwaldite.

Examples of phyllosilicate include calcium sodium borosilicate, calcium aluminum borosilicate, calcium titanium borosilicate, silver borosilicate, and zinc borosilicate.

For any of the above powders, under one embodiment, when the powder particles are mixed with the actinic light curable dip gel, the powder particles dissolve completely or partially. Under another embodiment, when mixed with the actinic light curable dip gel the powder particles are insoluble in the gel. The powder particles are considered to be insoluble when less than 5% of the diameter of the microsphere is lost to the solution. Generally, insoluble microspheres gain weight and size as they swell upon exposure to the gel and other ingredients.

After a desired number of repetitions, the nail is exposed to actinic light to cure the dip gel. The phrase “actinic light” refers to a light able to cause photochemical reactions. The phrase is interpreted broadly, including UV light and visible light.

A suitable actinic light may be natural sunlight. Another suitable actinic light may be a UV light lamp, such as a 36-watt lamp commonly used in many nail salons. Such a UV light lamp may operate at any wavelength required to cure the photopolymerizable composition, such as between 320 nm and 420 nm range at sufficient enough strength to cure the composition of the present invention.

The term “actinic light” includes any actinic light given off by an actinic light lamp that contains a fluorescent lamp, such as compact fluorescent light bulbs, or that give off an actinic light in the range of about 300 nm and about 700 nm. The term “actinic light lamp” also refers to a newer source of light or UV light radiation, such as a light-emitting diode lamp (commonly referred to as a “LED lamp”) that emits electromagnetic radiation in the range at a sufficiently high enough strength to cure the dip gel of the present invention. The term “actinic light lamp” also refers to any other type of source of light that comprises a light range at a sufficient enough strength to cure the dip gel composition of the present invention.

The exposure time should be long enough to allow for curing of the photopolymerizable dip gel composition. The exposure time may be in the range of about 5 seconds to about 6 minutes.

The present invention is also directed to a method of forming a nail cover comprising the steps of (a) applying a layer of the actinic light curable dip gel onto a nail; (b) applying a powder onto the nail; (c) exposing the nail to actinic light; and (d) repeating steps (a) to (c) 0 to 5 times. This method is particularly suitable for novice nail technicians.

The novice method exposes the nail to the actinic light after each application of actinic light curable dip gel layer. It has been found that even after the exposure to actinic light, the actinic light curable dip gel remains tacky enough that it picks up sufficient amount of polymer powder.

One of the advantages of the novice method is that this method is a more robust method of application. Another advantage of this method observed is that there is little or no need to file the nail each of the layers of the actinic light curable dip gel has been cured. Yet another advantage of the novice method is that this method lowers the possible damage to the layers as they are built up.

It has been observed that using the novice method requires a higher amount of layers to achieve the same degree of opacity and color saturation as in the method which exposes the layers to actinic light only once.

The present invention is also directed to a method of forming a nail cover comprising the steps of (a) applying a layer of the actinic light curable dip gel onto a nail; (b) exposing the nail to actinic light; (c) applying a powder onto the nail; and (d) repeating steps (a) to (c) 0 to 5 times.

This third method is similar to the novice method, except that steps (b) and (c) are switched. It has been found that after the exposure of the dip gel to the actinic light, the formed cured dip gel layer is tacky enough to allow for the powder to stick to the nail. Under this method, the powder then interacts with the dip gel that is applied to the nail subsequently.

The degree of tackiness is in large measure due to the completeness of the curing. The tackiness can be increased by decreasing the length of exposure time of the nail to the actinic light. Alternatively, the tackiness can be increased by decreasing the intensity of the actinic light.

The present invention is also directed to a method of forming a nail cover comprising the steps of (a)(i) applying a layer of the actinic light curable dip gel onto a nail; (ii) applying a powder onto the nail; (iii) repeating steps (i) and (ii) 0 to 5 times; (b) applying a top coat; and (c) exposing the nail to actinic light.

This fourth method is similar in various aspects to the above three methods, except that it differs from each of them in that the nail is exposed to the actinic light only after the application of a top coat. In this method, the top coat is applied to nail prior to curing of any of the gel layers.

One of the advantages of this method is the speed at which the nail covering can be made. This increased speed is due to the use of only a single exposure of the nail to the actinic light.

The exposure of the nails to the actinic light, as well as exposure of any tissue surrounding it, such as fingers and hands, is perceived by some clients to be hazardous to the client's health. This perception is common in cases where the actinic light is UV light. The single exposure of the nail to the actinic light is thus perceived by such clients to be less hazardous than repetitive exposures of the second and third method.

For each of the four methods disclosed above, additional steps may be performed by the technician to obtain the desired nail covering. These steps may follow the steps discussed above, or they may precede the steps discussed above, or they may occur between any of the steps discussed above.

Under one embodiment, prior to the application of the first layer of the actinic light curable dip gel onto a nail, the nail is coated with a nail polish primer or a nail polish base coat. Thus, the present invention is also directed to a method of forming a nail cover comprising the steps of (1) applying a layer of a nail polish primer or a nail polish base coat onto a nail; (2)(i) applying a layer of the actinic light curable dip gel onto a nail; (ii) introducing a polymer powder onto the nail; (iii) repeating steps (i) and (ii) 0 to 5 times; and (3) exposing the nail to actinic light.

Further, the present invention is also directed to a method of forming a nail cover comprising the steps of (1) applying a layer of a nail polish primer or a nail polish base coat onto a nail; (2)(i) applying a layer of the actinic light curable dip gel onto a nail; (ii) applying a powder onto the nail; (iii) repeating steps (i) and (ii) 0 to 5 times; and (3) exposing the nail to actinic light.

Also, the present invention is directed to a method of forming a nail cover comprising the steps of (1) applying a layer of a nail polish primer or a nail polish base coat onto a nail; (2) applying a layer of the actinic light curable dip gel onto a nail; (3) applying a powder onto the nail; (4) exposing the nail to actinic light; and (5) repeating steps (2) to (4) 0 to 5 times.

Further still, the present invention is also directed to a method of forming a nail cover comprising the steps of (1) applying a layer of a nail polish primer or a nail polish base coat onto a nail; (2) applying a layer of the actinic light curable dip gel onto a nail; (3) exposing the nail to actinic light; (4) applying a powder onto the nail; and (5) repeating steps (2) to (4) 0 to 5 times.

Moreover, the present invention is also directed to a method of forming a nail cover comprising the steps of (1) applying a layer of a nail polish primer or a nail polish base coat onto a nail; (2)(i) applying a layer of the actinic light curable dip gel onto a nail; (ii) applying a powder onto the nail; (iii) repeating steps (i) and (ii) 0 to 5 times; (3) applying a top coat; and (4) exposing the nail to actinic light.

The nail polish primer or a base coat may be any nail polish lacquer, including a lacquer that is made specifically to act as a nail polish primer, and a lacquer that the technical put down as the first coat onto a bare nail. Generally, the nail polish primer is an adhesion promoter that aids in bind the actinic light curable dip gel layer to the nail. The nail polish primer dries in less than 20 seconds. The nail polish primer typically extends the life of the manicure. The nail polish primer gives the actinic light curable dip gel an even surface to adhere to. By using a nail polish primer, the technician may prevent the nail covering from getting streaky and helps to eliminate ridges that sometimes appear on the client's nails. Further, the nail polish primer aids in preventing dark colors from staining the client's nail beds.

Under one embodiment, after the exposure of the nail to the actinic light, the nail coating is considered to be finished. Such nail coating does not need to be treated further.

Under an alternative embodiment, after the exposure of the nail to the actinic light, the nail coating is treated further. One such step is filing and/or buffing the nail until the surface is smooth. Thus, the present invention is also directed to a method of forming a nail cover comprising the steps of (1)(i) applying a layer of the actinic light curable dip gel of any of claims 1 to 32 onto a nail; (ii) applying a powder onto the nail; (iii) repeating steps (i) and (ii) 0 to 5 times; (2) exposing the nail to actinic light; and (3) filing and/or buffing the nail. The present invention is also directed to a method of forming a nail cover comprising the steps of (1) applying a layer of the actinic light curable dip gel onto a nail; (2) applying a powder onto the nail; (3) exposing the nail to actinic light; (4) repeating steps (1) to (3) 0 to 5 times; and (5) filing and/or buffing the nail. The present invention is also directed to a method of forming a nail cover comprising the steps of (1) applying a layer of the actinic light curable dip gel onto a nail; (2) exposing the nail to actinic light; (3) applying a powder onto the nail; (4) repeating steps (1) to (3) 0 to 5 times, and (5) filing and/or buffing the nail. The present invention is also directed to a method of forming a nail cover comprising the steps of (1)(i) applying a layer of the actinic light curable dip gel onto a nail; (ii) applying a powder onto the nail; (iii) repeating steps (i) and (ii) 0 to 5 times; (2) applying a top coat; (3) exposing the nail to actinic light; and (4) filing and/or buffing the nail.

Such polishing and/or buffing should be light enough not to file through the top powder layer and into the color layer.

Additional steps after the filing and/or buffing may be performed. This includes cleansing the nail with isopropanol to remove any debris from the surface of the buffed nail.

Under one embodiment, after the exposure of the nail to the actinic light, the nail coating is considered to be finished. Such a nail coating does not need to be treated further.

Under an alternative embodiment, after the exposure of the nail to the actinic light, the nail coating is treated further. One such step is the application of a top coat. Thus, the present invention is also directed to a method of forming a nail cover comprising the steps of (1)(i) applying a layer of the actinic light curable dip gel onto a nail; (ii) applying a powder onto the nail; (iii) repeating steps (i) and (ii) 0 to 5 times; (2) exposing the nail to actinic light; and (3) applying a layer of top coat. The present invention is also directed to a method of forming a nail cover comprising the steps of (1) applying a layer of the actinic light curable dip gel onto a nail; (2) applying a powder onto the nail; (3) exposing the nail to actinic light; (4) repeating steps (1) to (3) 0 to 5 times; and (5) applying a layer of top coat. The present invention is also directed to a method of forming a nail cover comprising the steps of (1) applying a layer of the actinic light curable dip gel of any of claims 1 to 32 onto a nail; (2) exposing the nail to actinic light; (3) applying a powder onto the nail; (4) repeating steps (1) to (3) 0 to 5 times; and (5) applying a layer of top coat. The present invention is also directed to a method of forming a nail cover comprising the steps of (1)(i) applying a layer of the actinic light curable dip gel onto a nail; (ii) applying a powder onto the nail; (iii) repeating steps (i) and (ii) 0 to 5 times; (2) applying a top coat; (3) exposing the nail to actinic light; and (4) applying a layer of top coat.

The phrases “top coat” and “top coat layer” refer to the coating that lies immediately atop of nail covering after exposing the nail to actinic light. In addition to the top coat layer, there may be more layers applied on top of the top coat. The top coat layer may also be an air dry layer.

Alternatively, the top coat layer is cured by actinic light radiation. The advantage of using an actinic light curable top coat, or top gel is that the finish has been observed to be much smoother, more durable, less dentable and less subject to cracking compared to other top coats. This durability is likely due to the equal hardness of all the layers as opposed to a traditional dip system where the underlayers are softer than the top most layers.

Under one embodiment, the top coat and any other coats over the cured nail cover are free of pigment so that the mirror appearance is clearly visible. Under another embodiment, the top coat or any other subsequent coats contain a small amount of pigment to yield a tinted top coat.

Additional steps after the application of top coat include washing hands to remove residual powder from the cuticle and finger tips.

For any of the methods described above, the nail technician may elect to complete each application on one hand at a time. Each step may be completed on every nail of one hand before moving to the next step. Under an alternative embodiment, one finger at a time may be completed before moving to another finger.

For any of the above-described methods, the “open” time is sufficiently long enough to allow the nail technician plenty of time to add the powder layer to the actinic light curable dip gel. The nail coat formed by the present invention is of sufficient quality that a single coat of the top coat may be required.

### EXPERIMENTAL

The formulations of two Examples are disclosed in Table 1. Both Examples are Working Examples.

TABLE 1
Actinic light curable dip gel formulation

| Ingredients | Example 1 | Example 2 |
|---|---|---|
| (Meth)acrylic copolymer | 19 | |
| Oligomer | | 37 |
| 2-Hydroxypropyl methacrylate | 44 | 19 |
| EGDMA, ethylene glycol dimethacrylate | 5 | |
| 3-Glycidyloxypropyltrimethoxysilane | | 10 |
| Photoinitiator | 3 | 3 |
| Acetate solvent | 29 | 30 |

All of the ingredients of actinic light curable dip gel formulations were obtained from commercial sources. The (meth)acrylic copolymer used is known for unique hardness versus flexibility and for excellent adhesion to various surfaces.

While the present invention has been described with reference to several embodiments, which embodiments have been set forth in considerable detail for the purposes of making a complete disclosure of the invention, such embodiments are merely exemplary and are not intended to be limiting or represent an exhaustive enumeration of all aspects of the invention. The scope of the invention is to be determined from the claims appended hereto. Further, it will be apparent to those of skill in the art that numerous changes may be made in such details without departing from the spirit and the principles of the invention.

What is claimed is:

1. An actinic light curable dip gel comprising:
(a) a film forming resin selected from the group consisting of poly(C₁₋₁₂alkyl(meth)acrylate) polymer, an acrylate functional acrylic resin, poly(meth)acrylate oligomer, styrene polymer, styrene divinylbenzene polymer, urethane polymer, cellulose acetate butyrate, cellulose acetate propionate, nitrocellulose, and mixtures thereof;
(b) a (meth)acrylate monomer; and
(c) one or more photoinitiators.

2. The actinic light curable dip gel of claim 1, wherein the film forming resin is selected from the group consisting of a poly(C₁₋₁₂alkyl(meth)acrylate) polymer, an acrylate functional acrylic resin, and mixtures thereof.

3. The actinic light curable dip gel of claim 1, wherein the actinic light curable dip gel comprises between 0 wt % and 50 wt % of a poly(C₁₋₁₂alkyl(meth)acrylate) polymer.

4. The actinic light curable dip gel of claim 1, wherein the actinic light curable dip gel comprises between 0 wt % and 50 wt % of an acrylate functional acrylic resin.

5. The actinic light curable dip gel of claim 1, wherein the actinic light curable dip gel comprises between 0 wt % and 50 wt % of a poly(meth)acrylate oligomer.

6. The actinic light curable dip gel of claim 1, wherein at least one of the one or more photoinitiators is selected from the group consisting of benzyl ketone, monomeric hydroxyl ketone, polymeric hydroxyl ketone, α-amino ketone, acyl phosphine oxide, metallocene, benzophenone, camphor quinone, and benzophenone derivatives.

7. The actinic light curable dip gel of claim 1, wherein at least one of the one or more photoinitiators is selected from the group consisting of 1-hydroxy-cyclohexylphenylketone; benzophenone; camphor quinone; 2-benzyl-2-(dimethylamino)-1-(4-(4-morphorlinyl)phenyl)-1-butanone; 2,2-dimethoxy-2-phenyl acetophenone; 2-methyl-1-(4-methylthio)phenyl-2-(4-morphorlinyl)-1-propanone; 2,4,6-trimethylbenzoyldiphenyl-phosphine oxide; bis(2,4,6-trimethyl benzoyl)phenyl wephosphine oxide; diphenyl-(2,4,6-trimethylbenzoyl) phosphine oxide; bis(2,6-dimethoxybenzoyl-2,4,4-trimethyl pentyl)phosphine oxide; 2-hydroxy-2-methyl-1-phenyl-propan-1-one; phenyl bis(2,4,6-trimethylbenzoyl) phosphine oxide; benzyl-dimethylketal; isopropylthioxanthone; bis(η⁵-2,4-cyclopentadien-1-yl)bis[2,6-difluoro-3-(1H-pyrrol-1-yl)phenyl]titanium), and mixtures of any of the foregoing.

8. The actinic light curable dip gel of claim 1, further comprising a solvent selected from the group consisting of C₁₋₄ alkyl acetate, C₁₋₃ alcohol, C₃₋₅ ketone, and mixtures thereof.

9. The actinic light curable dip gel of claim 1, further comprising at least one crosslinker.

10. The actinic light curable dip gel of claim 1, further comprising a crosslinker selected from the group consisting of tri(meth)acrylate, tetra(meth)acrylate, penta(meth)acrylate, ethylene glycol dimethylacrylate, EGDMA, di(ethylene glycol) dimethylacrylate, di-EGDMA, tri-(ethylene glycol) dimethylacrylate, and a mixture thereof.

11. The actinic light curable dip gel of claim 1, further comprising a crosslinker selected from the group consisting of trimethylol propane tri(meth)acrylate, ethoxylated glycerin tri(meth)acrylate, ethoxylated trimethylolpropane tri(meth)acrylate, ditrimethylol propane tetra(meth)acrylate, pentaerythritol tri(meth)acrylate, pentaerythritol tetra(meth)acrylate, propoxylated pentaerythritol tetra(meth)acrylate, ethoxylated pentaerythritol tetra(meth)acrylate, dipentaerythritol hexa(meth)acrylate, dipentaerythritol penta(meth)acrylate and ethoxylated iscyanuric acid tri(meth)acrylates.

12. The actinic light curable dip gel of claim 1, further comprising a silane coupling agent.

13. The actinic light curable dip gel of claim 1, wherein the gel is stable in a dark container at 49° C. for four months, or at 65° C. for 2 weeks.

14. A method of forming a nail cover comprising the steps of applying a layer of the actinic light curable dip gel of claim 1 onto a nail, applying a powder onto the nail, and exposing the nail to actinic light, wherein the steps may be carried out in any order.

15. The method of claim 14, wherein the powder is selected from an organic powder, inorganic powder, and a mixture thereof.

16. The method of claim 14, wherein the powder comprises a polymer powder selected from the group consisting of acrylic polymer, styrenic polymer, urethane polymer, and mixtures thereof.

17. The method of claim 16, wherein the polymer powder is crosslinked.

18. The method of claim 14, wherein the powder comprises particles selected from the group consisting of: a special effects pigment, spherical aliphatic polyurethane beads, micronized polyethylene and PTFE, micronized polypropylene wax, silicone resin powder, styrene polymer, styrene polymer, styrene copolymer, glass beads, nylon beads and polyethylene terephthalate based effect pigments, and mixtures thereof.

19. The method of claim 18, wherein the powder comprises special effects pigment selected from the group consisting of mica, aluminum, calcium borosilicate, titanium, synthetic mica, and a mixture thereof.

20. The method of claim 18, wherein the special effects pigment is selected from the group consisting a metal, a metal oxide, a main group oxide, a metal/main group oxide, and a mixture thereof.

21. The method of claim 14, wherein the powder is spherical.

22. The method of claim 14, wherein the mean particle size of poly(methyl methacrylate) particles is less than about 100 micrometers.

23. The method of the claim 14, wherein the first step is preceded by a step of applying a base coat layer, or a primer layer.

24. A method of forming a nail cover comprising the steps of
(a)(i) applying a layer of the actinic light curable dip gel of claim 1 onto a nail;
(ii) applying a powder onto the nail;
(iii) repeating steps (i) and (ii) 0 to 5 times; and
(b) exposing the nail to actinic light.

25. A method of forming a nail cover comprising the steps of
(a) applying a layer of the actinic light curable dip gel of claim 1 onto a nail;
(b) applying a powder onto the nail;
(c) exposing the nail to actinic light; and
(d) repeating steps (a) to (c) 0 to 5 times.

26. A method of forming a nail cover comprising the steps of
(a) applying a layer of the actinic light curable dip gel of claim 1 onto a nail;
(b) exposing the nail to actinic light;
(c) applying a powder onto the nail; and
(d) repeating steps (a) to (c) 0 to 5 times.

27. A method of forming a nail cover comprising the steps of
(a)(i) applying a layer of the actinic light curable dip gel of claim 1 onto a nail;
(ii) applying a powder onto the nail;
(iii) repeating steps (i) and (ii) 0 to 5 times;
(b) applying a top coat; and
(c) exposing the nail to actinic light.
